SEQUENCE LISTING (64 sequences)

SEQ ID NO: 1 - DNA, 372 bp, Homo sapiens
atggcccaggtgaaactgctcgagtcgggcccaggactggtgaagccttcggagaccctgtccctcacctgcactgtctctggtggctccatcagtagttattactggacctggattcgacagcccccagggaagggactggagtggattggatactcgtaccccagtgtgagcaccaagtacaacccctccctcaagagtcgagtctccatctcaacagacacgtccaagagccagttctccctgaagctgacttctgtgaccgctgcggacacggccgtttattactgtgcgcgaggagcagtgcgagctagtggactccatgacgcttatgacatctggggccaagggacactggtcaccgtctcttca

SEQ ID NO: 2 - protein, 129 aa, Homo sapiens
ACNVHMAQVKLLESGPGLVKPSETLSLTCTVSGGSISSYYWTWIRQPPGKGLEWIGYSYPSVSTKYNPSLKSRVSISTDTSKSQFSLKLTSVTAADTAVYYCARGAVRASGLHDAYDIWGQGTLVTVSS

SEQ ID NO: 3 - DNA, 375 bp, Homo sapiens
atggcacaggtgaaactgctcgagcagtcgggggcagggacgaggaagccttgggcgtccgtgaacctctcctgcaaggtttctggagtttccctcagaagttatgatttgcactggatgcgccagcccccaggaaaaagactggagtggatgggatggatgaacgccggcattggcaatacaaaatattcacagaacttgcagggcagagtcaccatttccagggactctacacgcacacaggctacaaggagatgtccgtgactgacatcggacgacacggttgtttattactgtgggaaagcaaacgttttattatgggtccgggaggtgtagtccatctggggccagggaacactggtcaccgtctcttca

SEQ ID NO: 4 - protein, 125 aa, Homo sapiens
MAQVKLLEQSGAGTRKPWASVNLSCKVSGVSLRSYDLHWMRQPPGKRLEWMGWMNAGIGNTKYSQNLQGRVTISRDSTRTQATRRCPCLTSDDTVVYYCGKANVLLWVREVESIWGQGTLVTVSS

SEQ ID NO: 5 - DNA, 369 bp, Homo sapiens
atggcccaggtgaaactgctcgagcagtctgggactgaggtgaagaagcctggggcctcagtgagggtctcctgcaaggcttctggattcaccttcaccgacgaatacatccactgggtgcgacaggcccctggacaagggcttgagtggatgggatggatcgaccctaaaactggtgacacaaagtattcacagaaacttcagggctgggtcaccatgaccagggacacgtccatcagcacagtctacatggaactgagcaggctgagatctgacgacacggccctctattactgtgcgagagagtcagctggtcatttggacgcttttgatatctggggccaagggacactggtcatcgtctcttcc

SEQ ID NO: 6 - protein, 123 aa, Homo sapiens
MAQVKLLEQSGTEVKKPGASVRVSCKASGFTFTDEYIHWVRQAPGQGLEWMGWIDPKTGDTKYSQKLQGWVTMTRDTSISTVYMELSRLRSDDTALYYCARESAGHLDAFDIWGQGTLVIVSS

SEQ ID NO: 7 - DNA, 372 bp, Homo sapiens
atggcccaggtgaaactgctcgagtcggggggaggcttggcacagccggggggatccctgagactctcctgtgcagcctcaggattcacctttttcatgcacggcatgagctgggtccgccaggctccagggagggggctggagtgggtctcaggtatccgtggaagtggtgatgagacatactacgcagactccgtgaagggccggttcagtatctccagagacaattccaagaacacggtatttctgcaaatgaacaggctgagagccgacgacacggccgtatattactgtgcgaaagattggcatagaggtgactggtacaggtggttcgacccgtggggccagggaaccctggtcactgtctcctca

SEQ ID NO: 8 - protein, 124 aa, Homo sapiens
MAQVKLLESGGGLAQPGGSLRLSCAASGFTFFMHGMSWVRQAPGRGLEWVSGIRGSGDETYYADSVKGRFSISRDNSKNTVFLQMNRLRADDTAVYYCAKDWHRGDWYRWFDPWGQGTLVTVSS

SEQ ID NO: 9 - DNA, 369 bp, Homo sapiens
atggcccaggtgaaactgctcgagtcgggcccaggactggtgaagccttcggagaccctgtccctcacctgcactgtctctggcgactccatcaccaatagtaattactagtgggaccggatcccccagtccccagggaaggggctggagtggattggaagtatctatcatagcgggttcaccgactacaacccgtccctcaagagtcgagtcacgatgtccgtagacacgtccaagaaccagttctccctgaaaatgagctctgtgaccgccgcagacacggctgtgatttactgtgcgagacggagtagtggccattatgatgcttttgatgtctggggccaagggacaaaggtcaccgtctcttca

SEQ ID NO: 10 - protein, 123 aa, Homo sapiens
MAQVKLLESGPGLVKPSETLSLTCTVSGDSITNSNYEWDRIPQSPGKGLEWIGSIYHSGFTDYNPSLKSRVTMSVDTSKNQFSLKMSSVTAADTAVIYCARRSSGHYDAFDVWGQGTKVTVSS

SEQ ID NO: 11 - DNA, 372 bp, Homo sapiens
atggccgaggtgcagctgctcgagtctgggggaggcctggtccagccgggggggtcccggagactctcctgtgcagcctctggattcaccgtcagtagtagctacatgagttgggtccgccagacacccgggaaggggctggagtggatttcagttttttacagtggaggaaccacatactacgcagacgccgtgaagggcagattcagcatctccatggacacttccaagaataccctgcatcttcaaatgaacagcctgagagtcgaggacacggctatctattactgtgcgagagttctaagtcgggctagtggaatgcctgatgccttcgatatttggggccccgggacaatggtcaccgtctcttca

SEQ ID NO: 12 - protein, 124 aa, Homo sapiens
MAEVQLLESGGGLVQPGGSRRLSCAASGFTVSSSYMSWVRQTPGKGLEWISVFYSGGTTYYADAVKGRFSISMDTSKNTLHLQMNSLRVEDTAIYYCARVLSRASGMPDAFDIWGPGTMVTVSS

SEQ ID NO: 13 - DNA, 360 bp, Homo sapiens
atggccgaggtgcagctgctcgagtcgggcccagggctggtgaagcctttggaaaccctgtccctcacttgcgctgtctctggtggctccatcactggttactactggagttggttccggcagcccccagggaagggactggagtggattgcgaacatctattatagtgggaatatcgactacagtccctccctcaggaatcgaatcaccgtttcgttggacacgtccaataaccacgcctccctaaagatcaatcgtgtgaccgtagcggacacggccacatattactgtgcgagaggtcgccccctgggaatgggttttgacccctggggccagggaaccctggtcctcgtctcatca

SEQ ID NO: 14 - protein, 120 aa, Homo sapiens
MAEVQLLESGPGLVKPLETLSLTCAVSGGSITGYYWSWFRQPPGKGLEWIANIYYSGNIDYSPSLRNRITVSLDTSNNHASLKINRVTVADTATYYCARGRPLGMGFDPWGQGTLVLVSS

SEQ ID NO: 15 - DNA, 360 bp, Homo sapiens
atggccgaggtgcagctgctcgagtcgggcccaggagtggtgaagccttcggagaccctgtccctcacctgcactgtctctggtggctcaatcactggttactactggagttggttccggcagaccccagggaagggactggaatggattacgaatatttattatagtggaaatatcgactacagcccctccctcaagagtcgaatcaccgtatcactagacacgtccaacaaccttgcctccctgaagctcacttccgtgaccgctgcggacacggccatgtattactgtgcgagaggtcgccccctggggatggggtttgacccctggggccagggaaccctggtcatcgtctcatca

SEQ ID NO: 16 - protein, 120 aa, Homo sapiens
MAEVQLLESGPGVVKPSETLSLTCTVSGGSITGYYWSWFRQTPGKGLEWITNIYYSGNIDYSPSLKSRITVSLDTSNNLASLKLTSVTAADTAMYYCARGRPLGMGFDPWGQGTLVIVSS

SEQ ID NO: 17 - DNA, 378 bp, Homo sapiens
atggccgaggtgcagctgctcgagcagtctggggctgaggtgaagaagcctggggccgcagtgaaggtttcctgtaaggcctctggatacaccttcaccacctatgcactgcattggctgcgccaggcccccggacaaaggcttgagtggatgggatggatcaacgctggcaatggtaacacagaatactcagagaagtttcagggcagaatcacccttacgagggacatgtccgcgaacacagcctacttggagctgaccaacctgagatctgaagacgcggctctatattattgtgcgagagatgaacgctcggttgtccaatattttgactggtaccttcaacactggggccagggaaccctggtcaccgtctca

SEQ ID NO: 18 - protein, 126 aa, Homo sapiens
MAEVQLLEQSGAEVKKPGAAVKVSCKASGYTFTTYALHWLRQAPGQRLEWMGWINAGNGNTEYSEKFQGRITLTRDMSANTAYLELTNLRSEDAALYYCARDERSVVQYFDWYLQHWGQGTLVTVS

SEQ ID NO: 19 - DNA, 381 bp, Homo sapiens
atggccgaggtgcagctgctcgagtctgggggaggcttggtacggccagggcggtccctagaactctcctgtacagcgtctggattcagctttcgtgattatgctttcacttgggtccgccaggctccaggcaaggggctggagtgggtaggttccattagacgcaaagcttatggtgagacaacagagtacgccgcgtctgtgaagggcagattcaccatctcaagagatgattccaaaagcatcgcctatctacaaatgaacagcctgagatccgaggactcagccgtctattactgtagtcgagttttgtcggttttggactactacttcgctatggacgtctggggccaagggaccacggtcaccgtctcctacagc

SEQ ID NO: 20 - protein, 127 aa, Homo sapiens
MAEVQLLESGGGLVRPGRSLELSCTASGFSFRDYAFTWVRQAPGKGLEWVGSIRRKAYGETTEYAASVKGRFTISRDDSKSIAYLQMNSLRSEDSAVYYCSRVLSVLDYYFAMDVWGQGTTVTVSYS

SEQ ID NO: 21 - DNA, 384 bp, Homo sapiens
atggcgctcgagcaggtccagcttgtgcagtctggggctgagttggagaagcctggggcctcagtgaaggtttcctgcaaggcttctggatacaccttcactacctatgctctgcattgggtgcgccaggcccccggacaaagccttgagtgggtgggatggatccaccctggcaatggcaacacagagttgtcacagaagttccagggcagagtctcctttaccagggacacttccgcgagcactgcctacatggaactgcgcagcctgagatctgaagacacggctctctattactgtgcgagagatgagcgttcggttgtacaacattttgactggttccttgaacactggggccagggaaccctggtcaccgtctcctca

SEQ ID NO: 22 - protein, 128 aa, Homo sapiens
MALEQVQLVQSGAELEKPGASVKVSCKASGYTFTTYALHWVRQAPGQSLEWVGWIHPGNGNTELSQKFQGRVSFTRDTSASTAYMELRSLRSEDTALYYCARDERSVVQHFDWFLEHWGQGTLVTVSS

SEQ ID NO: 23 - DNA, 393 bp, Homo sapiens
atggccgaggtgcagctgctcgagtctgggggaggcttggtcgagcctggagggtcgctgaggctctcctgtgccgcctctggattcaccttcagtcactactacttcaactggattcgccaggctccagggagggggctggagtgggtctcatatattagtagtggtggcggcggtaccatccactacgcagagtctgtgaagggccgattcaccatctctagagacaacgccaagaactctgtgtatctgcaaatgaacagcctgcgaaccgaggacacggccgtctattactgttcgagaggacagtattggtttgcctcgggaacttatcgggcgatcgatgatgcttctgatatttggggccaggggacaatggtcaccgtctcttca

SEQ ID NO: 24 - protein, 131 aa, Homo sapiens
MAEVQLLESGGGLVEPGGSLRLSCAASGFTFSHYYFNWIRQAPGRGLEWVSYISSGGGGTIHYAESVKGRFTISRDNAKNSVYLQMNSLRTEDTAVYYCSRGQYWFASGTYRAIDDASDIWGQGTMVTVSS

SEQ ID NO: 25 - DNA, 378 bp, Homo sapiens
atggccgaggtgcagctgctcgagtctgggggaggcttggtacagccagggcggtccctgggactaacctgtacaacttctggattcacgtttcgtgattatgccatgacctgggtccgccaggctccagggaaggggctggagtgggtaggctccattagacgcaaagcttatggtgggacaacagaatacgccgcgtctgtgaaagggagattcaccatctcaagagatgattccaaaagcatcgcctatctgcaaatgaacagcctgaaaaccgaggacacaggcgtgtatttctgtactcgagttttgtcggtcttggactactattacgggatggacgtctggggccaagggaccacggtcgccgtctcctca

SEQ ID NO: 26 - protein, 126 aa, Homo sapiens
MAEVQLLESGGGLVQPGRSLGLTCTTSGFTFRDYAMTWVRQAPGKGLEWVGSIRRKAYGGTTEYAASVKGRFTISRDDSKSIAYLQMNSLKTEDTGVYFCTRVLSVLDYYYGMDVWGQGTTVAVSS

SEQ ID NO: 27 - DNA, 381 bp, Homo sapiens
atggccgaggtgcagctgctcgagcagtcaggggctgaggtgaggaagcctggggcctcagtgaaggtttcctgcacggcttctggatacaccttcactacctttcctatacattggctgcgccaggcccccggacaaaggcttgagtggatgggatggatcaacgctggcaatggtaacaccgagtcttcacagaagttccagggcagagtcacctttaccagggacacatccgcgagcacagcctatatggagttgagcagcctgacatctgaagacacggctgtgtattactgtgcgagagatgagcgatcggttgtgcaacattttgactggttccttgagtattggggccagggaaccctggtcaccgtctcctca

SEQ ID NO: 28 - protein, 127 aa, Homo sapiens
MAEVQLLEQSGAEVRKPGASVKVSCTASGYTFTTFPIHWLRQAPGQRLEWMGWINAGNGNTESSQKFQGRVTFTRDTSASTAYMELSSLTSEDTAVYYCARDERSVVQHFDWFLEYWGQGTLVTVSS

SEQ ID NO: 29 - DNA, 1620 bp, Human metapneumovirus
atgtcttggaaagtggtgatcattttttcattgttaataacacctcaacacggtcttaaagagagctacttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagagctcagaacagtttctgctgatcaactggcaagagaggagcaaattgagaatcccagacaatctagattcgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagagagctgaaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggtttggaatcccgataggagtttacggaagctccgtaatttacatggtgcaactgccaatctttggggttatagacacgccttgctggatagtaaaagcagccccttcttgctcagaaaaaaagggaaactatgcttgcctcttaagagaagatcaaggatggtattgtcagaatgcagggtcaactgtttactacccaaatgaaaaagactgtgaaacaagaggagaccatgtcttttgcgacacagcagcaggaatcaatgtcgctgagcagtcaaaggagtgcaacatcaacatatccactactaattacccatgcaaagttagcacaggaagacatcctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaagtttcctgaagatcaattcaatgttgcacttgaccaagttttcgagagcattgagaacagtcaggccttggtggatcaatcaaacagaatcctaagcagtgcagagaaaggaaacactggcttcatcattgtaataattctaattgctgtcctaggctctaccatgatcctagtgagtgtttttatcataataaagaaaacaaagaaacccacaggagcacctccagagctgagtggtgtcacaaacaatggcttcataccacataattag

SEQ ID NO: 30 - protein, 539 aa, Human metapneumovirus
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGIPIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 31 - DNA, 1620 bp, Human metapneumovirus
atgagctggaaggtggtgattatcttcagcctgctgattacacctcaacacggcctgaaggagagctacctggaagagagctgctccaccatcaccgagggctacctgagcgtgctgcggaccggctggtacaccaacgtgttcaccctggaggtgggcgacgtggagaacctgacctgcagcgacggccctagcctgatcaagaccgagctggacctgaccaagagcgctctgagagagctgaagaccgtgtccgccgaccagctggccagagaggaacagatcgagaaccctcggcagagcagattcgtgctgggcgccatcgctctgggagtcgccgctgccgctgcagtgacagctggagtggccattgctaagaccatcagactggaaagcgaggtgacagccatcaacaatgccctgaagaagaccaacgaggccgtgagcaccctgggcaatggagtgagagtgctggccacagccgtgcgggagctgaaggacttcgtgagcaagaacctgaccagagccatcaacaagaacaagtgcgacatcgatgacctgaagatggccgtgagcttctcccagttcaacagacggttcctgaacgtggtgagacagttctccgacaacgctggaatcacacctgccattagcctggacctgatgaccgacgccgagctggctagagccgtgcccaacatgcccaccagcgctggccagatcaagctgatgctggagaacagagccatggtgcggagaaagggcttcggcatcctgattggggtgtatggaagctccgtgatctacatggtgcagctgcccatcttcggcgtgatcgacacaccctgctggatcgtgaaggccgctcctagctgctccgagaagaaaggaaactatgcctgtctgctgagagaggaccagggctggtactgccagaacgccggaagcacagtgtactatcccaacgagaaggactgcgagaccagaggcgaccacgtgttctgcgacaccgctgccggaatcaacgtggccgagcagagcaaggagtgcaacatcaacatcagcacaaccaactacccctgcaaggtgagcaccggacggcaccccatcagcatggtggctctgagccctctgggcgctctggtggcctgctataagggcgtgtcctgtagcatcggcagcaatcgggtgggcatcatcaagcagctgaacaagggatgctcctacatcaccaaccaggacgccgacaccgtgaccatcgacaacaccgtgtaccagctgagcaaggtggagggcgagcagcacgtgatcaagggcagacccgtgagctccagcttcgaccccatcaagttccctgaggaccagttcaacgtggccctggaccaggtgtttgagaacatcgagaacagccaggccctggtggaccagagcaacagaatcctgtccagcgctgagaagggcaacaccggcttcatcattgtgatcattctgatcgccgtgctgggcagctccatgatcctggtgagcatcttcatcattatcaagaagaccaagaaacccaccggagcccctcctgagctgagcggcgtgaccaacaatggcttcattccccacaactga

SEQ ID NO: 32 - protein, 539 aa, Human metapneumovirus
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVAAAAAVTAGVAIAKTIRLESEVTAINNALKKTNEAVSTLGNGVRVLATAVRELKDFVSKNLTRAINKNKCDIDDLKMAVSFSQFNRRFLNVVRQFSDNAGITPAISLDLMTDAELARAVPNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 33 - DNA, 1620 bp, Human metapneumovirus
atgtcttggaaagtgatgatcatcatttcgttactcataacaccccagcacgggctaaaggagagttatttggaagaatcatgtagtactataactgagggatacctcagtgttttaagaacaggctggtacactaatgtcttcacattagaagttggtgatgttgaaaatcttacatgtactgatggacctagcttaatcaaaacagaacttgacctaacaaaaagtgctttaagggaactcaaaacagtctctgctgatcagttagcgagagaggagcaaattgaaaatcccagacaatcaagatttgtcctaggtgcaatagctctcggagttgctacagcagcagcagtcacagcaggtattgcaatagccaaaaccataaggcttgagagtgaggtgaatgcaattaaaggtgctctcaaacaaactaatgaagcagtatccacattaggaaatggtgtgcgggtcctagccactgcagtgagagagctgaaagaatttgtgagcaaaaacctgactagtgcaatcaacaggaacaaatgtgacattgctgatctgaagatggctgtcagcttcagtcaattcaacagaagatttctaaatgttgtgcggcagttttcagacaatgcagggataacaccagcaatatcattggacctaatgactgatgctgagttggccagagctgtatcatacatgccaacatctgcaggacagataaaactgatgttggagaaccgcgcaatggtaaggagaaaaggatttggaatcctgataggggtctacggaagctctgtgatttacatggttcaattgccgatctttggtgtcatagatacaccctgttggataatcaaggcagctccctcttgctcagaaaaaaacgggaattatgcttgcctcctaagagaggatcaagggtggtattgtaaaaatgcaggatccactgtttactacccaaatgaaaaagactgcgaaacaagaggtgatcatgttttttgtgacacagcagcagggatcaatgttgctgagcaatcaagagaatgcaacatcaacatatctactaccaactacccatgcaaagtcagcacaggaagacaccctataagcatggttgcactatcacctctcggtgctttggtggcttgctataaaggggtaagctgctcgattggcagcaatcgggttggaatcatcaaacaattacctaaaggctgctcatacataaccaaccaggatgcagacactgtaacaattgacaataccgtgtatcaactaagcaaagttgaaggtgaacagcatgtaataaaagggaggccagtttcaagcagttttgatccaatcaggtttcctgaggatcagttcaatgttgcgcttgatcaagtcttcgaaagcattgagaacagtcaggcactagtggaccagtcaaacaaaattctaaacagtgcagaaaaaggaaacactggtttcattattgtaataattttggttgctgttcttggtttaaccatgatttcagtgagcatcatcatcataatcaagaaaacaaggaagcccacaggagcacctccagagctgaatggtgtcaccaacggcggtttcataccacatagttag

SEQ ID NO: 34 - protein, 539 aa, Human metapneumovirus
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVDQSNKILNSAEKGNTGFIIVIILVAVLGLTMISVSIIIIIKKTRKPTGAPPELNGVTNGGFIPHS

SEQ ID NO: 35 - DNA, 1620 bp, Human metapneumovirus
atgtcttggaaagtgatgattatcatttcgttactcataacacctcagcacggactaaaagaaagttatttagaagaatcatgtagtactataactgaaggatatctcagtgttttaagaacaggttggtacaccaatgtctttacattagaagttggtgatgttgaaaatcttacatgtactgatggacctagcttaatcaaaacagaacttgacctaaccaaaagtgctctaagagaactcaaaacagtttctgctgatcagttagcgagagaagaacaaattgagaatcccagacaatcaaggtttgtcctaggtgcaatagctcttggtgttgccacagcagcagcagtcacagcaggcattgcgatagccaaaaccataaggcttgagagtgaagtgaatgcaatcaaaggtgctctcaaaacaaccaacgaggcagtatccacactaggaaatggagtgcgagtcctagccaccgcagtaagagagctgaaagaatttgtgagcaaaaacctgactagtgcaattaacaagaacaaatgtgacattgctgatctgaagatggctgtcagcttcagtcaattcaacagaagattcctaaatgttgtgcggcagttttcagacaatgcagggataacaccagcaatatcattggacctaatgactgatgctgagctggccagagctgtatcatacatgccaacatctgcaggacagataaaactaatgttagagaaccgtgcaatggtgaggagaaaaggatttggaatcttgataggggtctacggaagctccgtgatttacatggtccagctgccgatctttggtgtcatagatacaccttgttggataatcaaggcagctccctcttgttcagaaaaagatggaaattatgcttgcctcctaagagaggatcaagggtggtattgtaaaaatgcaggatccactgtttactacccaaatgataaagactgcgaaacaagaggtgatcatgttttttgtgacacagcagcagggatcaatgttgctgagcaatcaagagaatgcaacatcaacatatctacaaccaactacccatgcaaagtcagcacaggaagacaccctatcagcatggttgcactatcacctctcggtgctttggtagcttgctacaaaggggttagctgttcgattggcagtaatcgggttggaataatcaaacaactacctaaaggctgctcatacataactaaccaggacgcagacactgtaacaattgacaacactgtgtatcaactaagcaaagttgagggtgaacagcatgtaataaaagggagaccagtttcaagcagtttcgatccaatcaagtttcctgaggatcagttcaatgttgcgcttgatcaagtctttgaaagcattgaaaacagtcaagcactagtggaccagtcaaacaaaattctgaacagtgcagaaaaaggaaacactggcttcattattgtaataattttgattgctgttcttgggttaaccatgatttcagtgagcatcatcatcataatcaaaaaaacaaggaaacccacaggggcacctccagagctgaatggtgttaccaacggcggttttataccgcatagttag

SEQ ID NO: 36 - protein, 539 aa, Human metapneumovirus
MSWKVMIIISLLITPQHGLKKLFLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 37 - DNA, 318 bp, Homo sapiens
atggccgagctccagccgccctcagtgtccgtgtctccaggacagacagccaggatcacctgctctggagagaatttgggaaaaaaatatgtttgttggtatcagcggaagccaggccagtcccctgtcttggtcatgtatgaagattctaagcggccctcagggatccctgaacgattctctggctccaattctgggaccacagccactctgaccatcagcgggacgcaggccatggatgaggctgactatttctgtcaagtgtgggtcagcagcaccgaggttcttttcggcggcgggaccaagctgtccgtccta

SEQ ID NO: 38 - protein, 106 aa, Homo sapiens
MAELQPPSVSVSPGQTARITCSGENLGKKYVCWYQRKPGQSPVLVMYEDSKRPSGIPERFSGSNSGTTATLTISGTQAMDEADYFCQVWVSSTEVLFGGGTKLSVL

SEQ ID NO: 39 - DNA, 324 bp, Homo sapiens
atggccgagctcacggcagttccagacaccttgtctttgtctccaggggaaagagccaccctctcctgcagggccagtcagagtcttaccagctccttcttagcctggtaccagcagaaacctggccaggctcccaggttcctcatctatgatgcatccatcagggccactggcgtcccagacaggttcagtggcagtgggtctgggacagacttcactctcaccatcagcagactggagcctgaagattttgcagtctattactgtcagcagtatggtagctcaccaaggacgttcggccaagggaccaaggtggaagtgaaa

SEQ ID NO: 40 - protein, 108 aa, Homo sapiens
MAELTAVPDTLSLSPGERATLSCRASQSLTSSFLAWYQQKPGQAPRFLIYDASIRATGVPDRFSGSGSGTDFTLTISRLEPEDFAVYYCQQYGSSPRTFGQGTKVEVK

SEQ ID NO: 41 - DNA, 318 bp, Homo sapiens
atggccgagctcacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagttctatcactttcggccctgggaccaaagtggatatcaaa

SEQ ID NO: 42 - protein, 106 aa, Homo sapiens
MAELTQSPSSLSASVGDRVTITCRASQSISSYLNWYQQKPGKAPKLLIYAASSLQSGVPSRFSGSGSGTDFTLTISSLQPEDFATYYCQQSYSSITFGPGTKVDIK

SEQ ID NO: 43 - DNA, 330 bp, Homo sapiens
atggccgagctccctcagccccactctgtgtcggagtctccggggaagacggtagtcatctcctgcacccgaagtagtggcaagattgacagcaactatgtgcagtggtaccggcggcgcccgggcagttcccccactgttgtgatctttgaggatgaccaaagaccctctggggtccctgatcgattctctggctccatcgacaggtcctccaattctgcctccctcaccatttctggactgatggctgaggacgaggctgactattactgtcagtcttatgatgcccgctatcaggtgttcggcggagggaccaagctgaccgtcctg

SEQ ID NO: 44 - protein, 110 aa, Homo sapiens
MAELPQPHSVSESPGKTVVISCTRSSGKIDSNYVQWYRRRPGSSPTVVIFEDDQRPSGVPDRFSGSIDRSSNSASLTISGLMAEDEADYYCQSYDARYQVFGGGTKLTVL

SEQ ID NO: 45 - DNA, 321 bp, Homo sapiens
atggccgagctcactcagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaacctgtggacgttcggccaagggaccaaggtggaaatcaaa

SEQ ID NO: 46 - protein, 107 aa, Homo sapiens
MAELTQSPSSLSASVGDRVTITCRASQSISSYLNWYQQKPGKAPKLLIYAASSLQSGVPSRFSGSGSGTDFTLTISSLQPEDFATYYCQQSYSNLWTFGQGTKVEIK

SEQ ID NO: 47 - DNA, 318 bp, Homo sapiens
gagctcgccctgattcagcctgcctccgtgtccgtgtccccaggacagacagccagcatcacctgctctggagataaattgggggataaatatgcttcctggtatcagcagaagccaggccagtcccctgtgctggtcatctatcaagatagcgagcggccctcagggatccctgagcgattctctggctccaactctgggaacacagccactctgaccatcagcgggacccaggctatggatgaggctgactattactgtcaggcgtgggacagcagcactgcggtattcggcggagggaccacgctgaccgtccta

SEQ ID NO: 48 - protein, 106 aa, Homo sapiens
ELALIQPASVSVSPGQTASITCSGDKLGDKYASWYQQKPGQSPVLVIYQDSERPSGIPERFSGSNSGNTATLTISGTQAMDEADYYCQAWDSSTAVFGGGTTLTVL

SEQ ID NO: 49 - DNA, 327 bp, Homo sapiens
gagctcacgcagccgccctcagtgtctgggaccccagggcagagggtcaccatctcctgcactgggagcagctccaacatcgggacaccttatgatgtacactggtatcagcaactcccaggaacagcccccaaactcctcatctatggtgacaccaatcggccctcaggggtccctgaccgattctctggctccaagtctggcacgtcagcctccctggccatcactgggctccaggctgaggatgagggtgattattactgccagtcgtatgacagcagcctgagtggttgggtattcggcggagggaccaagctgaccgtccta

SEQ ID NO: 50 - protein, 109 aa, Homo sapiens
ELTQPPSVSGTPGQRVTISCTGSSSNIGTPYDVHWYQQLPGTAPKLLIYGDTNRPSGVPDRFSGSKSGTSASLAITGLQAEDEGDYYCQSYDSSLSGWVFGGGTKLTVL

SEQ ID NO: 51 - DNA, 330 bp, Homo sapiens
gagctcgccctgactcagcctccctccgtgtctgggtctcctggacagtcgatcaccatctcctgcactggaaccagcagtgacgttggtggttataactttgtctcctggtaccaacaccacccaggcaaagcccccaaactcatgatttatgatgtcagtcttcggccctcaggggtttctaatcgcttctctggctccaagtctggcaatacggcctccctgaccatctctgggctccaggctgaggacgaggctgattattactgcagctcatatacaggcagcagcactgtgcttttcggcggagggaccaagctgaccgtctta

SEQ ID NO: 52 - protein, 110 aa, Homo sapiens
ELALTQPPSVSGSPGQSITISCTGTSSDVGGYNFVSWYQHHPGKAPKLMIYDVSLRPSGVSNRFSGSKSGNTASLTISGLQAEDEADYYCSSYTGSSTVLFGGGTKLTVL

SEQ ID NO: 53 - DNA, 321 bp, Homo sapiens
gagctcgtgatgacccagtctccatcctccctgtctgcatctgtaggagacaccatcaccctcacttgccgggcgagccagggcattggcgagtctttagcctggtatcagcagagtccggggaaagcccctaaactcctcctctctgctgcatccagattggaaagtggggtcccgtccaggttcagtggcagtgggtctgggtcggattacactctcaccatcaacagcctgcagcctgaagattttgcaatgtattactgtcaacagtattttgggactcctctcactttcggcggagggaccaagctggagatcaaa

SEQ ID NO: 54 - protein, 107 aa, Homo sapiens
ELVMTQSPSSLSASVGDTITLTCRASQGIGESLAWYQQSPGKAPKLLLSAASRLESGVPSRFSGSGSGSDYTLTINSLQPEDFAMYYCQQYFGTPLTFGGGTKLEIK

SEQ ID NO: 55 - DNA, 330 bp, Homo sapiens
gagctcgccctgactcagcctccctccgtgtctgggtctcctggacagtccatcaccatctcctgcactggaaccagcaatgatgttgggagttctaaccttgtctcctggtaccaacaacacccaggcaaagcccccaaactcatgattcatgaggccagtaagcggccctcaggggtttctaatcgcttctctggctccaagtctggcaacacggcctccctgacaatctctgggctccaggctgaggacgaggctgattattactgtagctcatatgcaggcagtagcactgtggtattcggcggagggaccaaactgaccgtccta

SEQ ID NO: 56 - protein, 110 aa, Homo sapiens
ELALTQPPSVSGSPGQSITISCTGTSNDVGSSNLVSWYQQHPGKAPKLMIHEASKRPSGVSNRFSGSKSGNTASLTISGLQAEDEADYYCSSYAGSSTVVFGGGTKLTVL

SEQ ID NO: 57 - DNA, 324 bp, Homo sapiens
gagctcacactcacgcagtctccaggcaccctgtctttgtctccgggggaaagagccaccctctcctgcagggccagtcagagtgttggcaagtcttatttagcctggtaccagcagaaacctggccaggctcccaggctcctcctctatgggtcatccaacagggccactggcatcccagacaggttcagtggtagtgggtctgggacagacttcactctcaccatcaacagactggcgcctgaagatttcgcagtctatttctgtcagcaatatggttcctcacctgcgacgttcggccaagggaccaaggtggagatcaag

SEQ ID NO: 58 - protein, 108 aa, Homo sapiens
ELTLTQSPGTLSLSPGERATLSCRASQSVGKSYLAWYQQKPGQAPRLLLYGSSNRATGIPDRFSGSGSGTDFTLTINRLAPEDFAVYFCQQYGSSPATFGQGTKVEIK

SEQ ID NO: 59 - DNA, 333 bp, Homo sapiens
gagctcactcagtctccactctccctgcccgtcacccttgggcagccggcctccatctcctgcaggtctagtgaaagcctcctatacactgatggcaacacctacttgaattggttccagcagaggccaggccaatctccaaggcgcctactttatcaggtttctaaccgggaccctggggtcccagacagattccgcggcagtgggtcaggcactgatttcacactgaggatcagcagggtggaggctgaggatgttgggacttattattgcatgcaagctacacactggcctccgtacacttttggccaggggacgaagctggagatcaat

SEQ ID NO: 60 - protein, 111 aa, Homo sapiens
ELTQSPLSLPVTLGQPASISCRSSESLLYTDGNTYLNWFQQRPGQSPRRLLYQVSNRDPGVPDRFRGSGSGTDFTLRISRVEAEDVGTYYCMQATHWPPYTFGQGTKLEIN

SEQ ID NO: 61 - DNA, 330 bp, Homo sapiens
gagctcgtgttgacgcagccgccctcagtgtctgcggccccaggacagaaggtcaccatctcctgctctggaaacagctccaacattgagagtaattacgtatcctggtaccagcagctttcaggcagagcccccaaactcctcatttatgacaataacaagcgaccctcagggatccctgagcgattctctggctccaagtctggcacgtctgccaccctgggcgtcaccggactccagcctggggacgaggccgattattactgcgcaacatgggataacagcctgagtgctggggtgttcggcggagggaccaggctgaccgtccta

SEQ ID NO: 62 - protein, 110 aa, Homo sapiens
ELVLTQPPSVSAAPGQKVTISCSGNSSNIESNYVSWYQQLSGRAPKLLIYDNNKRPSGIPERFSGSKSGTSATLGVTGLQPGDEADYYCATWDNSLSAGVFGGGTRLTVL

SEQ ID NO: 63 - DNA, 321 bp, Homo sapiens
gagctcgtgatgacccagtctccttcatccctgtctcaatttgttggaggcagagtcaccatcacttgccgggcgagccagcacattggcaattctttagcctggtatcagcagagtccagggaaagcccccaaactcctcctctctgatacgtccaaattacagagtggggtcccatcccgctttagtggcagtgggtctgggccggattacactctcaccatcaacaacgtgcagcctgaagattttggagtctattactgtcaacagtattttgggactcctctcactttcggcggggggaccaagcttgagatcaaa

SEQ ID NO: 64 - protein, 107 aa, Homo sapiens
ELVMTQSPSSLSQFVGGRVTITCRASQHIGNSLAWYQQSPGKAPKLLLSDTSKLQSGVPSRFSGSGSGPDYTLTINNVQPEDFGVYYCQQYFGTPLTFGGGTKLEIK

(19) United States
(12) Patent Application Publication
(10) Pub. No.: US 2011/0135645 A1
(43) Pub. Date: Jun. 9, 2011

(54) HUMAN ANTIBODIES NEUTRALIZING HUMAN METAPNEUMOVIRUS

(75) Inventors: R. Anthony Williamson, La Jolla, CA (US); Zhifeng Chen, Vista, CA (US); Pietro Paolo Sanna, San Diego, CA (US); Dennis R. Burton, La Jolla, CA (US); James Crowe, Nashville, TN (US); John V. Williams, Nashville, TN (US)

(73) Assignee: The Scripps Research Institute, La Jolla, CA (US)

(21) Appl. No.: 12/311,587

(22) PCT Filed: Oct. 4, 2007
(86) PCT No.: PCT/US07/80498

§ 371 Date: Dec. 2, 2009

Related U.S. Application Data
(60) Provisional application No. 60/849,626, filed on Oct. 4, 2006.
(60) Provisional application No. 60/918,030, filed on Mar. 13, 2007.

(51) Int. Cl. A61K 39/395; C07K 16/10; G01N 33/68; C12Q 1/70; A61P 31/14; A61P 37/04

(52) U.S. Cl. 424/137.1; 530/387.5; 530/391.3; 530/387.3; 436/501; 424/159.1; 435/5

(57) ABSTRACT

The present invention discloses methods for generating antibodies to human metapneumovirus (HMPV) polypeptides, including antibodies that immunospecifically bind to a HMPV F-protein. The invention also discloses methods for preventing, treating, or ameliorating symptoms associated with HMPV infection.

### BACKGROUND OF THE INVENTION

1. Field of the Invention

The present invention relates generally to antibodies, and more specifically to antibodies or fragments thereof that specifically bind to human metapneumovirus (HMPV) polypeptides and methods for preventing, treating, or ameliorating symptoms associated with HMPV infection.

2. Background Information

Human metapneumovirus (HMPV) is a recently discovered respiratory pathogen now known to be a major global cause of serious respiratory disease in young children, the elderly, and immunocompromised individuals. Clinically, HMPV respiratory disease is highly analogous to that caused by human respiratory syncytial virus (HRSV), and the two pathogens are closely related.

Human metapneumovirus was first described in 2001, having been isolated from children presenting with symptoms of acute respiratory disease with an undetermined etiology. Serological studies in the Netherlands with archival patient samples indicate that the virus has been circulating in humans for at least the past 50 years. Further, seroprevalence analysis indicates that the virus infects over 50% of infants by age 2 and almost all children one or more times before the age of 5 years. Intensive study since its discovery has detected HMPV in patient samples across the globe and has identified the pathogen as a major cause (second only to HRSV) of acute respiratory tract disease in infants and adults. As is the case with HRSV, HMPV infection rates vary seasonally in temperate climates, peaking during the early to mid-winter months and extending into early spring.

Although the full extent of disease burden manifested through HMPV infection has yet to be formally determined, it is estimated that HMPV accounts for roughly 5 to 15% of respiratory disease in hospitalized young children. Within this group, children under 2 years of age appear at most risk for serious disease following HMPV infection. In adult populations, the elderly and immunocompromised are particularly prone to problems following HMPV infection. For example, HMPV was found in elderly individuals with chronic obstructive pulmonary disease (COPD) in the absence of other known pathogens. HMPV has also been shown to cause severe and sometimes fatal respiratory tract disease in adults and children with hematologic malignancies. There is also evidence that HMPV infection may lead to or exacerbate asthmatic conditions. Further, HMPV has been suggested as a co-pathogen in a subset of severe acute respiratory syndrome caused by the SARS coronavirus, and as a cofactor for pathogenesis in the case of fatal encephalitis.

Typically, HMPV infected patients present with a spectrum of disease that is highly similar to that seen with HRSV infection, although the observed frequencies of given symptoms vary between the particular HMPV patient cohorts. HMPV infections of both the lower and upper respiratory tract in children are also associated with a 12 to 50% incidence of concomitant otitis media. Exacerbations leading to particularly severe respiratory tract disease were observed in some children co-infected with HMPV and HRSV, but not in others. Therefore, although co-infections with HMPV and HRSV are not likely to be uncommon given their prevalence and overlapping winter epidemics, it presently remains unclear whether or not synergistic pathology can occur between these two viruses. Importantly, polymerase chain reaction (PCR) based diagnosis of active HMPV infection determined that the virus was rarely present in infants or adults without symptoms indicative of respiratory disease, suggesting an absence of asymptomatic or subclinical HMPV infection in these two groups.

HMPV has been assigned to the Metapneumovirus genus of the subfamily Pneumoviriniae, family, Paramyxoviridae and order Mononegavirales. The virus is most closely related to avian metapneumovirus (AMPV), the only other member of the Metapneumovirus genus, that is the causative agent of severe rhinotracheitis in turkeys, but also infects chickens and pheasants, and to HRSV which is assigned within the Pneumovirus genus, the other genus of the Pneumoviriniae family.

As with other pneumoviruses, the G- and F-proteins direct the infection process. The G-glycoprotein, possessing the features of a type II mucin-like molecule, but lacking the cluster of cysteine residues found in its HRSV and AMPV homologues, helps mediate virus attachment of the target cell receptor, with the F-glycoprotein promoting fusion of the viral envelope membrane with the host cell membrane, thus, facilitating access of the viral RNA into the target cell cytoplasm. However, HMPV lacking the G- and surface protein gene (SH) proteins, replicates successfully in the African green monkey (non-human primate host) suggesting that the viral attachment function can ultimately be performed by another viral protein.

Human metapneumovirus (HMPV) F-protein is thought to be a major antigenic determinant that mediates effective neutralization and protection against HMPV infection. The HMPV F-protein is a major antigenic determinant that can mediate extensive cross-lineage neutralization and protection. Production of MAbs to the HMPV F-protein is critical for development of diagnostic techniques, vaccine research, and studies on viral pathogenesis.

### SUMMARY OF THE INVENTION

The present invention is based on the development of an antibody or fragment thereof that specifically binds to a human metapneumovirus virus (HMPV) F-protein antigen.

In one embodiment, an isolated human antibody is disclosed that specifically binds to a human metapneumovirus (HMPV) fusion glycoprotein (F-protein). In one aspect, the F-protein is selected from the group consisting of an amino acid sequence as set forth in SEQ ID NO:30, SEQ ID NO:32, SEQ ID NO: 34, and SEQ ID NO:36. In a related aspect, the antibody neutralizes HMPV genogroups A1, A2, B1, and B2.

In another aspect, the antibody comprises an HCDR3 amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28.

In another embodiment, a method for identifying a neutralizing antibody is disclosed including generating a panel of antibodies against recombinant, immature, and mature forms of a fusion protein (F-protein), comparing the binding of the antibodies to each form of F-protein by competition analysis, determining the K_{d} for each antibody in the panel against each form of the F-protein, identifying one or more antibodies in the panel whose K_{d} is one or more orders of magnitude higher for the recombinant or immature form of the F-protein than the mature form of the F-protein, and determining the neutralizing efficiency of the one or more antibodies identified, where a neutralizing antibody has lower binding constant for mature forms of the F-protein than a non-neutralizing antibody. In a related aspect, the method includes analyzing the one or more antibodies identified by microneutralization assay. In one embodiment, a method of treating a respiratory condition in a subject is disclosed, where the condition is caused by a human metapneumovirus (HMPV) infection, comprising administering to the subject an antibody which neutralizes HMPV.

In another embodiment, a vaccine is disclosed comprising one or more human antibodies that specifically binds to a human metapneumovirus (HMPV) fusion glycoprotein (F-protein). In a related aspect, the vaccine neutralized HMPV genogroups A1, A2, B1, and B2.

In one embodiment, a method of diagnosing a metapneumovirus (HMPV) infection is disclosed, including contacting a sample from a subject with a human antibody that specifically binds to a HMPV fusion glycoprotein (F-protein) under conditions which allow for antibody/F-protein complex formation, contacting the sample with a reagent that interacts with the antibody, and detecting the interaction of the reagent with the antibody, where detection of the reagent-antibody interaction is indicative of the presence of an MHPV infection in the subject. In a related aspect, the subject is human. In another related aspect, the reagent is an antibody directed against the human antibody that specifically binds to the HMPV F-protein.

In another embodiment, a diagnostic kit is disclosed for determining the presence of human metapneumovirus (HMPV) in a sample, including a device for contacting a biological sample with one or more human antibodies that specifically bind to one or more HMPV fusion glycoproteins (F-proteins) under conditions that allow for the formation of a complex between the one or more antibodies and one or more HMPV F-proteins, one or more reagents which remove non-complexed antibody, one or more reagents that recognize the antibody, instructions which provide procedures on the use of the antibody and reagents, and a container which houses the one or more antibodies, reagents, and instructions.

Various combinations of the foregoing embodiments are contemplated by the present invention, as are embodiments including other aspects as recited below.

### BRIEF DESCRIPTION OF THE DRAWINGS

FIG. 1 shows immunofluorescent images of HMPV-infected LLC-MK2 cell monolayers stained with an Fab generated phage display against HMPV FΔTM protein. Secondary detection is accomplished with goat anti-human Fab. Left panel, 10× magnification. Right panel, 20× magnification (shows two syncytia).

FIG. 2 shows light microscopic and immunofluorescent images of HMPV-infected LLC-MK2 cell monolayers stained with human anti-HMPV F Fab and AlexaFluor568-goat antihuman IgG. (A), (B). Fab ACN044. (C), (D). Fab DSλ7. 20× magnification.

FIG. 3 shows surface plasmon resonance analysis of DSλ7 Fab. (A). Association/disassociation curves of decreasing concentrations of DSλ7 against immobilized HMPV FΔTM protein. Palivizumab (RSV F-specific MAb) was used as an irrelevant control. (B). Association/disassociation curve of DSλ7 at 100 nM concentration against immobilized HMPV FΔTM protein and RSV FΔTM protein.

FIG. 4 graphically illustrates nasal titer of HMPV shed 4 days post-infection from animal strains and species tested (top), and lung titer of HMPV shed 4 days post-infection from animal strains and species tested (bottom). (A) Guinea pigs; (B) C3H mice; (C) CBA mice; (D) C57BI/10 mice; (E) SJL mice; (F) BALB/c mice; (G) 129 mice; (H) AKR mice; (I) DBA/1 mice; (J) DBA/2 mice; (K) Syrian golden hamsters; and (L) Cotton rats.

FIG. 5 graphically illustrates the kinetics of HMPV shedding in cotton rats. Animals were infected intranasally and sacrificed at 2, 4, 6, 8, 10, and 14 days post-infection. Closed circles=nose, open circles=lung.

FIG. 6 shows the histopathology of HMPV infection in cotton rat lungs. (A): at lower power, the control lung is free of interstitial infiltrates, with normal airways (H&E×35). (B): the interstitium of the HMPV-infected lung is expanded by mononuclear cells (H&E×25). (C): higher magnification of the uninfected lung does not show interstitial infiltrates or peribronchiolar inflammation (H&E×62.5). (D): higher magnification of HMPV-infected lung shows hypersecretory changes of the epithelium and peribronchiolar mononuclear cell infiltrate (H&E×125).

FIG. 7 shows immunohistochemistry of HMPV in cotton rat lungs. (A): control lung is negative for HMPV antigen, with minimal background (×250). (B): HMPV-infected lung. HMPV antigen is detected at the luminal surface of ciliated cells in a discontinuous pattern (arrows). Note mixed inflammatory cells in the lumen (L). (×250).

FIG. 8 graphically illustrates nasal and lung HMPV titers of previously mock infected of HMPV-infected animals following challenge 21 days after primary inoculation (Left) and serum HMPV-neutralizing antibody titers of previously mock-infected of HMPV-infected animals following challenge 21 days after primary inoculation (Right).

FIG. 9 shows nasal (A) and lung (B) titers of HMPV. Groups are as defined in the Example section. Tissue virus titers were log(10)-transformed for statistical analysis. Comparisons between groups were made using Wilcoxon rank sum test. Horizontal bars represent geometric mean; dotted line indicates limit of detection (5 PFU/g).

FIG. 10 shows dose response relationship of DSλ7. (A) Nasal titers of HMPV. (B) Lung titers of HMPV. Groups are as defined in the text. Linear regression was used to examine a dose-response relationship between Fab DSλ7 and log(10)-transformed viral titer as described in the Example section. Dotted line indicates limit of detection (5 PFU/g).

### DETAILED DESCRIPTION OF THE INVENTION

Before the present composition and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, exemplar methods and materials are now described.

“Single-chain antigen-binding-protein” refers to a polypeptide composed of an immunoglobulin light-chain variable region amino acid sequence (V_{L}) tethered to an immunoglobulin heavy-chain variable region amino acid sequence (V_{H}) by a peptide that links the carboxyl terminus of the V_{L} sequence to the amino terminus of the V_{H} sequence.

“Single-chain antigen-binding-protein-coding gene” refers to a recombinant gene coding for a single-chain antigen-binding-protein.

“Polypeptide and peptide” refer to a linear series of amino acid residues connected one to the other by peptide bonds between the alpha-amino and carboxy groups of adjacent residues.

“Protein” refers to a linear series of greater than about 50 amino acid residues connected one to the other as in a polypeptide.

“Immunoglobulin product” refers to a polypeptide, protein, or multimeric protein containing at least the immunologically active portion of an immunoglobulin heavy chain and is thus capable of specifically combining with an antigen. Exemplary immunoglobulin products are an immunoglobulin heavy chain, immunoglobulin molecules, substantially intact immunoglobulin molecules, any portion of an immunoglobulin that contains the paratope, including those portions known in the art as Fab fragments, Fab′ fragment, Fab2′ fragment, and Fv fragment.

“Immunoglobulin molecule” refers to a multimeric protein containing the immunologically active portions of an immunoglobulin heavy chain and immunoglobulin light chain covalently coupled together and capable of specifically combining with antigen.

“Fab fragment” refers to a multimeric protein consisting of the portion of an immunoglobulin molecule containing the immunologically active portions of an immunoglobulin heavy chain and an immunoglobulin light chain covalently coupled together and capable of specifically combining with antigen. Fab fragments are typically prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using methods well known in the art.

“Fv fragment” refers to a multimeric protein consisting of the immunologically active portions of an immunoglobulin heavy chain variable region and an immunoglobulin light chain variable region covalently coupled together and capable of specifically combining with antigen. Fv fragments are typically prepared by expressing in suitable host cell the desired portions of immunoglobulin heavy chain variable region and immunoglobulin light chain variable region using methods well known in the art.

“Immunoglobulin superfamily molecule” refers to a molecule that has a domain size and amino acid residue sequence that is significantly similar to immunoglobulin or immunoglobulin related domains. The significance of similarity is determined statistically using a computer program such as the Align program described by Dayhoff et al., Meth Enzymol. 91: 524-545 (1983). A typical Align score of less than 3 indicates that the molecule being tested is a member of the immunoglobulin gene superfamily.

“Epitope” refers to a portion of a molecule that is specifically recognized by an immunoglobulin product. It is also referred to as the determinant or antigenic determinant.

“Neutralize” refers to an activity of an antibody, where the antibody can inhibit the infectivity of a virus or the toxicity of a toxin molecule.

“Genogroup” refers to strains of viruses which comprise as set of closely related genes that code for the same or similar proteins.

Like HRSV and AMPV, HMPV is an enveloped virus containing a genome of approximately 13 kilobases comprised of negative-strand RNA. The organization is compact, with approximately 95% of the genome represented in the predicted open reading frames. There are thought to be 8 genes that occur in the following order in the 3′ to 5′ direction: the nucleocapsid RNA binding-protein gene ((N): e.g., GenBank Accession Nos. DQ841210; DQ841209; DQ841208; DQ841207; DQ834376); the nucleocapsid phosphoprotein gene ((P): e.g., GenBank Accession Nos. DQ112319; DQ112318; DQ112317; DQ112316; DQ112315; DQ112314); the non-glycosylated matrix protein gene ((M): e.g., GenBank Accession Nos. DQ439961; DQ439960; DQ439959; DQ439958; DQ439957); the fusion glycoprotein gene ((F): e.g., including but not limited to, SEQ ID NO:30; SEQ ID NO:32; SEQ ID NO:34; SEQ ID NO:36; the transcription elongation factor gene ((M2-1): e.g., GenBank Accession Nos. AAS22126; AAS22086; AAS22118; AAS22110; AAS22102); the small hydrophobic surface protein gene ((SH): e.g., GenBank Accession No. AAS22088; AAS22128; AA22120; AAS22112; AAS22104); the major attachment protein gene ((G): e.g., GenBank Accession Nos. AAS22129; AAS22121; AAS22113; AAS22105; AAS22097); and major polymerase subunit gene ((L): e.g., GenBank Accession Nos. AY550173; AY550172; AY550171; AY550170; AY550169). A ninth protein, the RNA synthesis regulatory factor ((M2-2): e.g., GenBank Accession Nos. AAS22095; AAS22103; AAS22111; AAS22119; AAS22127) is predicted, arising from a second overlapping open reading frame within the M2 gene sequence as in HRSV (van den Hoogen et al., Nat Med (2001) 7:719-724). The SH molecule, predicted to be a type II glycoprotein, also inserts into the virus envelop via a hydrophobic signal anchor sequence that is located near its amino terminus.

Phylogenetic scrutiny of partial or complete sequences of one or more of the F-, G-, SH-, N-, P-, M-, M-2- and L-genes from many HMPV isolated with broad geographic and temporal distribution has been performed (Bastien et al., J Clin Microbiol (2004) 42:3532-3537; Biacchesi et al., Virol (2003) 315:1-9; Ishiguro et al., Clin Microbiol (2004) 42:3406-3414). Data sets from these analyses consistently identify two HMPV genetic lineages, termed A and B, which is similar to the groupings for isolates of HRSV. HMPV gene sequences within groups A and B can be further divided into 2 clades per group, denoted A 1 and A2, and B1 and B2. It is apparent that these 4 HMPV subtypes can circulate simultaneously within the same geographical area, and that the relative proportions of each subtype can vary from one season to the next. When comparing the predicted amino acid sequences between HMPV isolates assigned to group A or to group B, the G- and SH-proteins possessed low amino acid identities (33% and 58% identity, respectively). In contrast, the other HMPV gene products were highly conserved; F-protein 94-95% amino acid identity; N-protein 95-96% identity; P-protein 85% identity; M-protein 97% identity; M2-1 95-96% identity; M2-2-protein 89-90% identity; and L-protein 94% identity. This clear pattern between HMPV groups of more conserved sequence for the F and non-envelop viral proteins, and greater diversity in the G- and SH-proteins, generally mirrors that seen with the equivalent analysis in HRSV. The exception is M2-2 protein, which is markedly more conserved between HMPV strains than between strains of HRSV. Further, it seems that, at the amino acid level, the F-protein of HMPV was 6% less divergent between phylogenetic groups than the HRSV F-protein.

In one embodiment, a method of mammalian protein expression to generate a soluble form of the HMPV F protein (FΔTM) that was highly immunogenic and induced neutralizing antibodies in cotton rats is disclosed. This construct was used to select fully human MAbs from combinatorial phage display libraries. In one aspect, this approach is effective in isolating numerous human Fabs that bind to HMPV-infected cells. In another aspect, the FΔTM protein retains neutralizing epitopes present on the native F protein.

The degree of genetic variability between individual HMPV strains, and particularly the genes encoding the three envelop glycoproteins, will have a direct influence upon antigenic diversity in the infected host. Analogous to HRSV, it seems that the antibody response against the F-protein of HMPV is cross-reactive, and cross-protective between phylogenetic groups A and B, whereas the immune response against G-protein tends to be group specific and generally unable to provide cross-clade neutralization and protection. While not being bound by theory, from the phylogenetic studies of HMPV isolates, it would be reasonable to conclude that the group A and group B virus isolates could also represent distinct antigenic groupings, and that most of the antigenic diversity, as the genetic diversity, could arise from the G- and SH-proteins.

In humans, reinfection by paramyxoviruses, including HMPV, occurs throughout life, even with genetically homologous viruses. Under these circumstances, it is thus difficult to meaningfully determine the most significant corollary of genetic and antigenic diversity in HMPV: whether or not viruses of one genetic group induce greater immunological protection against infection by homologous strains, rather than heterologous strains. It is highly unlikely, however, that the high degree of divergence of the G-protein between and within HMPV subgroups will contribute to limited cross-neutralizing and cross-protecting antibody responses against the G-protein, therefore implicating the invariant F-protein as the major operational target for HMPV neutralizing antibodies.

In one embodiment, an isolated antibody is disclosed that specifically binds to a human metapneumovirus (HMPV) fusion glycoprotein (F-protein). In one aspect, the F-protein is selected from the group consisting of an amino acid sequence as set forth in SEQ ID NO:30, SEQ ID NO:32, SEQ ID NO: 34, and SEQ ID NO:36. In a related aspect, the antibody neutralizes HMPV genogroups A1, A2, B1, and B2.

Antigenic diversity provides a tactical advantage to a pathogen. The sequence diversity observed for the G-protein between phylogenetic groups of HMPV is largely confined to the extracellular portions of the molecules suggesting, as postulated for similar changes in the G-protein of HRSV, that this phenomenon is an evolutionary response to immunologic pressure. This property will likely reduce the probability that antibodies against G-protein of one HMPV subgroup will cross-neutralize virus belonging to other subgroups. In combination, these factors make the G-protein an unattractive molecule to target in antibody prophylaxis of HMPV infections. This is unlikely the case for the F-protein.

F-protein is highly conserved across HMPV isolates, and there is little sign of antigenic drift over time possibly reflecting that greater functional and structural constraints apply to the amino acid substitutions in this molecule than in the G-protein. Referring to the HRSV, the F-protein is a major target for cross-strain neutralizing and protective antibodies. Thus, the F-protein of HMPV represents a highly favorable target for antibody prophylaxis and also as a component of candidate vaccines.

The F-protein is synthesized initially as an inactive monomeric precursor F₀, the protein is cleaved into two subunits (F₁ and F₂) that are linked covalently via disulfide bonds. Four of the F₁-F₂ molecules interact via the F₁ subunit to form the mature viron spike.

In one embodiment, the present invention discloses a method for identifying a neutralizing antibody against F-protein of HMPV, including generating a panel of antibodies against recombinant, immature, and mature forms of a fusion protein (F-protein), comparing the binding of the antibodies to each form of F-protein by competition analysis, determining the K_{d} for each antibody in the panel against each form of the F-protein, identifying one or more antibodies in the panel whose K_{d} is one or more orders of magnitude higher for the recombinant or immature form of the F-protein than the mature form of the F-protein, and determining the neutralizing efficiency of the one or more antibodies as identified, where a neutralizing antibody has lower binding constant for mature forms of the F-protein than a non-neutralizing antibody. In a related aspect, the F-protein is in an oligomeric form.

“Oligomer” refers to any substance or type of substance that is composed of molecules containing a small number—typically two to about ten—of constitutional units in repetitive covalent or non-covalent linkage; the units may be of one or of more than one species.

The usefulness of a method of the invention to produce functional polypeptides, including functional protein complexes, is exemplified herein by the production of functional antibodies. The term “antibody” is used broadly herein to refer to a polypeptide or a protein complex that can specifically bind an epitope of an antigen. Generally, an antibody contains at least one antigen binding domain that is formed by an association of a heavy chain variable region domain and a light chain variable region domain, particularly the hypervariable regions.

The construction of libraries of fragments of antibody molecules that are expressed on the surface of filamentous bacteriophage and the selection of phage antibodies by binding to antigens have been recognized as powerful means of generating new tools for research and clinical applications. This technology, however, has been mainly used to generate phage antibodies specific for purified antigens that are available in sufficient quantities for solid-phase dependent selection procedures. The effectiveness of such phage antibodies in biochemical and functional assays varies; typically, the procedure used to select determines their utility.

Typically, many antigens of interest are not available in pure form in very large quantities. This can limit the utility of phage antibodies in binding such materials for research and clinical applications. Further, the utility of phage antibodies in such applications is directly proportional to the purity of the antigens and purification methods to assure the specificity of the isolated phage antibodies. Human monoclonal antibodies that bind to native cell surface structures are expected to have broad application in therapeutic and diagnostic procedures. An important extension of phage antibody display technology is a strategy for the direct selection of specific antibodies against antigens expressed on the surface of subpopulations of cells present in a heterogenous mixture. Such antibodies may be derived from a single highly-diverse display library (see, e.g., U.S. Pat. No. 6,265,150, herein incorporated by reference).

Display libraries (i.e., from bacteriophage, particularly filamentous phage, and especially phage M13, Fd, and F1) involve inserted libraries encoding polypeptides to be displayed into either gIII or gVIII of these phage forming a fusion protein. See, e.g., Dower, WO 91/19818; Devlin, WO 91/18989; MacCafferty, WO 92/01047 (gene III); Huse, WO 92/06204; Kang, WO 92/18619 (gene VIII). Such a fusion protein comprises a signal sequence, usually from a secreted protein other than the phage coat protein, a polypeptide to be displayed and either the gene III or gene VIII protein or a fragment thereof. Exogenous coding sequences are often inserted at or near the N-terminus of gene III or gene VIII although other insertion sites are possible. Some filamentous phage vectors have been engineered to produce a second copy of either gene III or gene VIII. In such vectors, exogenous sequences are inserted into only one of the two copies. Expression of the other copy effectively dilutes the proportion of fusion protein incorporated into phage particles and can be advantageous in reducing selection against polypeptides deleterious to phage growth. In another variation, exogenous polypeptide sequences are cloned into phagemid vectors which encode a phage coat protein and phage packaging sequences but which are not capable of replication. Phagemids are transfected into cells and packaged by infection with helper phage. Use of phagemid systems also has the effect of diluting fusion proteins formed from coat protein and displayed polypeptide with wild type copies of coat protein expressed from the helper phage. See, e.g., Garrard, WO 92/09690. Eukaryotic viruses can be used to display polypeptides in an analogous manner.

Spores can also be used as display packages. In this case, polypeptides are displayed from the outer surface of the spore. For example, spores from B. subtilis have been reported to be suitable. Sequences of coat proteins of these spores are known in the art. Cells can also be used as display packages. Polypeptides to be displayed are inserted into a gene encoding a cell protein that is expressed on the cells surface. Bacterial cells include, but are not limited to, Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and Escherichia coli. Details of outer surface proteins are well known in the art (see, e.g., Ladner, et al., U.S. Pat. No. 5,571,698). For example, the lamB protein of E. coli is suitable.

Antibody chains can be displayed in single or double chain form. Single chain antibody libraries can comprise the heavy or light chain of an antibody alone or the variable domain thereof. However, more typically, the members of single-chain antibody libraries are formed from a fusion of heavy and light chain variable domains separated by a peptide spacer within a single contiguous protein. See e.g., Ladner, et al., WO 88/06630; McCafferty, et al., WO 92/01047. Double-chain antibodies are formed by noncovalent association of heavy and light chains or binding fragments thereof. Double chain antibodies can also form by association of two single chain antibodies, each single chain antibody comprising a heavy chain variable domain, a linker and a light chain variable domain. In such antibodies, known as diabodies, the heavy chain of one single-chain antibody binds to the light chain of the other and vice versa, thus forming two identical antigen binding sites. Thus, phage displaying single chain antibodies can form diabodies by association of two single chain antibodies as a diabody.

The diversity of antibody libraries can arise from obtaining antibody-encoding sequences from a natural source, such as a nonclonal population of immunized or unimmunized B cells. Alternatively, or additionally, diversity can be introduced by artificial mutagenesis of nucleic acids encoding antibody chains before or after introduction into a display vector. Such mutagenesis can occur in the course of PCR or can be induced before or after PCR.

Nucleic acids encoding antibody chains to be displayed optionally flanked by spacers are inserted into the genome of a phage as discussed above by standard recombinant DNA techniques (see generally, Sambrook, et al., Molecular Cloning, A Laboratory Manual, 2d ed., Cold Spring Harbor Laboratory Press, Cold Spring Harbor, N.Y., 1989, incorporated by reference herein). The nucleic acids are ultimately expressed as antibody chains (with or without spacer or framework residues). In phage, bacterial and spore vectors, antibody chains are fused to all or part of the an outer surface protein of the replicable package. Libraries often have sizes of about 10³, 10⁴, 10⁶, 10⁷, 10⁸, or more members.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR).

In general, the nucleotides comprising a polynucleotide are naturally occurring deoxyribonucleotides, such as adenine, cytosine, guanine or thymine linked to 2′ deoxyribose, or ribonucleotides such as adenine, cytosine, guanine or uracil linked to ribose. Depending on the use, however, a polynucleotide also can contain nucleotide analogs, including non naturally occurring synthetic nucleotides or modified naturally occurring nucleotides. Nucleotide analogs are well known in the art and commercially available (e.g., Ambion, Inc.; Austin Tex.), as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

The term “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element. A recombinant nucleic acid molecule also can be based on, but manipulated so as to be different, from a naturally occurring polynucleotide, for example, a polynucleotide having one or more nucleotide changes such that a first codon, which normally is found in the polynucleotide or such that a sequence of interest is introduced into the polynucleotide, for example, a restriction endonuclease recognition site or a splice site, a promoter, a DNA origin of replication, or the like.

As used herein, the term “operatively linked” means that two or more molecules are positioned with respect to each other such that they act as a single unit and effect a function attributable to one or both molecules or a combination thereof. For example, a polynucleotide encoding a polypeptide can be operatively linked to a transcriptional or translational regulatory element, in which case the element confers its regulatory effect on the polynucleotide similarly to the way in which the regulatory element would effect a polynucleotide sequence with which it normally is associated within a cell.

A polynucleotide of the invention also can be flanked by a first cloning site and a second cloning site, thus providing a cassette that readily can be inserted into or linked to a second polynucleotide. Such flanking first and second cloning sites can be the same or different, and one or both independently can be one of a plurality of cloning sites, i.e., a multiple cloning site. A vector of the invention also can contain one or more additional nucleotide sequences that confer desirable characteristics on the vector, including, for example, sequences that facilitate manipulation of the vector. As such, the vector can contain, for example, one or more cloning sites, for example, a cloning site, which can be a multiple cloning site, positioned such that a heterologous polynucleotide can be inserted into the vector and operatively linked to the first promoter. The vector also can contain a prokaryote origin of replication (ori), for example, an E. coli ori or a cosmid ori, thus providing a vector which can be passaged in a prokaryote host cell for DNA amplification.

Double-chain antibody display libraries represent a species of the display libraries discussed above. Production of such libraries is well known in the art. For example, in double-chain antibody phage display libraries, one antibody chain is fused to a phage coat protein, as is the case in single chain libraries. The partner antibody chain is complexed with the first antibody chain, but the partner is not directly linked to a phage coat protein. Either the heavy or light chain can be the chain fused to the coat protein. Whichever chain is not fused to the coat protein is the partner chain. This arrangement is typically achieved by incorporating nucleic acid segments encoding one antibody chain gene into either gIII or gVIII of a phage display vector to form a fusion protein comprising a signal sequence, an antibody chain, and a phage coat protein. Nucleic acid segments encoding the partner antibody chain can be inserted into the same vector as those encoding the first antibody chain. Optionally, heavy and light chains can be inserted into the same display vector linked to the same promoter and transcribed as a polycistronic message. Alternatively, nucleic acids encoding the partner antibody chain can be inserted into a separate vector (which may or may not be a phage vector). In this case, the two vectors are expressed in the same cell (see WO 92/20791). The sequences encoding the partner chain are inserted such that the partner chain is linked to a signal sequence, but is not fused to a phage coat protein. Both antibody chains are expressed and exported to the periplasm of the cell where they assemble and are incorporated into phage particles.

The display vector can be designed to express heavy and light chain constant regions or fragments thereof in-frame with heavy and light chain variable regions expressed from inserted sequences. Typically, the constant regions are naturally occurring human constant regions; a few conservative substitutions can be tolerated. In a Fab fragment, the heavy chain constant region usually comprises a C_{H}1 region, and optionally, part or all of a hinge region, and the light chain constant region is an intact light chain constant region, such as C_{κ} or C_{λ}. Choice of constant region isotype depends in part on whether complement-dependent cytotoxity is ultimately required. For example, human isotypes IgG1 and IgG4 support such cytotoxicity whereas IgG2 and IgG3 do not. Alternatively, the display vector can provide nonhuman constant regions. In such situations, typically, only the variable regions of antibody chains are subsequently subcloned from display vectors and human constant regions are provided by an expression vector in frame with inserted antibody sequences.

Antibody encoding sequences can be obtained from lymphatic cells of a human (see, Examples, infra). Polynucleotides useful for practicing a method of the invention can be isolated from cells producing the antibodies of interest, for example, B cells from an immunized subject or from an individual exposed to a particular antigen, can be synthesized de novo using well known methods of polynucleotide synthesis, or can be produced recombinantly. In one aspect, antibody libraries of the present invention are prepared from bone marrow lymphocytes of different adult donors, wherein the donors have been exposed to HMPV or infected by HMPV at least once in their lifetime. In another aspect, immunized human lymphocytes can be immortalized with infection with Epstein-Barr virus to generate monoclonal antibody secreting cultures.

Rearranged immunoglobulin genes can be cloned from genomic DNA or mRNA. For the latter, mRNA is extracted from the cells and cDNA is prepared using reverse transcriptase and poly dT oligonucleotide primers. Primers for cloning antibody encoding such sequences are well known in the art.

Repertoires of antibody fragments have been constructed by combining amplified V_{H} and V_{L} sequences together in several ways. Light and heavy chains can be inserted into different vectors and the vectors combined in vitro or in vivo. Alternatively, the light and heavy chains can be cloned sequentially into the same vector or assembled together by PCR and then inserted into a vector. Repertoires of heavy chains can also be combined with a single light chain or vice versa.

Typically, segments encoding heavy and light antibody chains are subcloned from separate populations of heavy and light chains resulting in random association of a pair of heavy and light chains from the populations in each vector. Thus, modified vectors typically contain combinations of heavy and light chain variable region not found in naturally occurring antibodies. Some of these combinations typically survive the selection process and also exist in the polyclonal libraries.

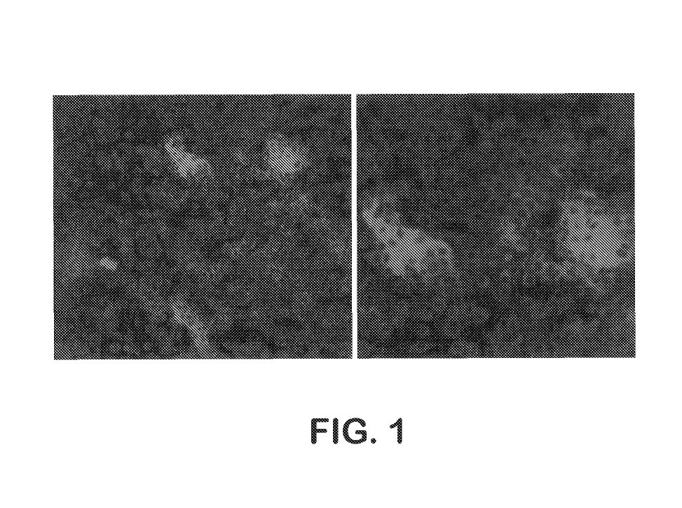

Some exemplary vectors and procedures for cloning populations of heavy chain and light chain encoding sequences have been described by Huse, WO 92/06204. Diverse populations of sequences encoding H_{C} polypeptides are cloned into M13IX30 and sequences encoding L_{C} polypeptides are cloned into M13IX11. The populations are inserted between the XhoI-SeeI or StuI restriction enzyme sites in M13IX30 and between the SacI-XbaI or EcoRV sites in M13IX11 (FIGS. 1A and B of Huse, respectively). Both vectors contain two pairs of MluI-HindIII restriction enzyme sites (FIGS. 1A and B of Huse) for joining together the H_{C} and L_{C} encoding sequences and their associated vector sequences. The two pairs are symmetrically oriented about the cloning site so that only the vector proteins containing the sequences to be expressed are exactly combined into a single vector.

Others exemplary vectors and procedures for cloning antibody chains into filamentous phage are described in U.S. Pat. No. 6,794,132, herein incorporated by reference. In general, a vector of the invention can be a circularized vector, or can be a linear vector, which has a first end and a second end. A linear vector of the invention can have one or more cloning sites at one or both ends, thus providing a means to circularize the vector or to link the vector to a second polynucleotide, which can be a second vector that is the same as or different from the vector of the invention. The cloning site can include a restriction endonuclease recognition site (or a cleavage product thereof), a recombinase site, or a combination of such sites.

The vector can further contain one or more expression control elements, for example, transcriptional regulatory elements, additional translational elements, and the like. In one embodiment, the vector contains an initiator ATG codon operatively linked to the sequence encoding a promoter, such that a polynucleotide encoding a polypeptide can be operatively linked adjacent to an initiation ATG codon. Accordingly, the vector also can contain a cloning site that is positioned to allow operative linkage of at least one heterologous polynucleotide to such an ATG codon. A vector of the invention also can contain a nucleotide sequence encoding a first polypeptide operatively linked to the first promoter wherein the encoding nucleotide sequence is modified to contain one or more cloning sites, including, for example, upstream of and near the ATG codon, downstream of and near the ATG codon, and/or at or near the C-terminus of the encoded polypeptide. Such a vector provides a convenient means to insert a nucleotide sequence encoding a second polypeptide therein, either by substitution of the nucleotide sequence encoding the first polypeptide, or in operative linkage near the N-terminus or C terminus of the encoded polypeptide such that a fusion protein comprising the first and second polypeptide can be expressed.

One of the most useful aspects of using a recombinant expression system for antibody production is the ease with which the antibody can be tailored by molecular engineering. This allows for the production of antibody fragments and single-chain molecules, as well as the manipulation of full-length antibodies. For example, a side range of functional recombinant-antibody fragments, such as Fab, Fv, single-chain and single-domain antibodies, may be generated. This is facilitated by the domain structure of immunoglobulin chains, which allows individual domains to be “cut and spliced” at the gene level.

Polynucleotides encoding humanized monoclonal antibodies, for example, can be obtained by transferring nucleotide sequences encoding mouse complementarity determining regions from heavy and light variable chains of the mouse immunoglobulin gene into a human variable domain gene, and then substituting human residues in the framework regions of the murine counterparts. General techniques for cloning murine immunoglobulin variable domains are known in the art.

The methods of the invention also can be practiced using polynucleotides encoding human antibody fragments isolated from a combinatorial immunoglobulin library. Cloning and expression vectors that are useful for producing a human immunoglobulin phage library can be obtained, for example, from Stratagene Cloning Systems (La Jolla, Calif.).

A polynucleotide encoding a human monoclonal antibody also can be obtained, for example, from transgenic mice that have been engineered to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody secreting hybridomas, from which polynucleotides useful for practicing a method of the invention can be obtained. Methods for obtaining human antibodies from transgenic mice are known in the art.

The polynucleotide also can be one encoding an antigen binding fragment of an antibody. Antigen binding antibody fragments, which include, for example, Fv, Fab, Fab′, Fc, and F(ab′)2 fragments, are well known in the art, and were originally identified by proteolytic hydrolysis of antibodies. For example, antibody fragments can be obtained by pepsin or papain digestion of whole antibodies by conventional methods. Antibody fragments produced by enzymatic cleavage of antibodies with pepsin generate a 5S fragment denoted F(ab′)2. This fragment can be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see, for example, Goldenberg, U.S. Pat. No. 4,036,945 and U.S. Pat. No. 4,331,647, each of which is incorporated by reference).

Another form of an antibody fragment is a peptide coding for a single complementarity-determining region (CDR). CDR peptides can be obtained by constructing a polynucleotide encoding the CDR of an antibody of interest, for example, by using the polymerase chain reaction to synthesize the variable region from RNA of antibody-producing cells (see, for example, Larrick et al., Methods: A Companion to Methods in Enzymology 2:106, 1991, which is incorporated herein by reference). Polynucleotides encoding such antibody fragments, including subunits of such fragments and peptide linkers joining, for example, a heavy chain variable region and light chain variable region, can be prepared by chemical synthesis methods or using routine recombinant DNA methods, including phage display, beginning with polynucleotides encoding full length heavy chains and light chains, which can be obtained as described above. In one aspect, the antibody of the present invention comprises an HCDR3 amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20; SEQ ID NO:22, SEQ ID NO:24, SEQ ID NO:26, or SEQ ID NO:28.

In one embodiment, a multimeric protein is a Fab fragment consisting of a portion of an immunoglobulin heavy chain and a portion of an immunoglobulin light chain. The immunoglobulin heavy and light chains are associated with each other and assume a conformation having an antigen binding site specific for a preselected or predetermined antigen. The antigen binding site on a Fab fragment has a binding affinity or avidity similar to the antigen binding site on an immunoglobulin molecule.

Genes useful in practicing this invention include genes coding for a polypeptide contained in immunoglobulin products, immunoglobulin molecules, Fab fragments, and Fv fragments. These include genes coding for immunoglobulin heavy and light chain variable regions. Typically, the genes coding for the immunoglobulin heavy chain variable region and immunoglobulin light chain variable region of an immunoglobulin capable of binding a preselected antigen are used.

These genes are isolated from cells obtained from a mammal, in one aspect, a human, which has been immunized with an antigenic ligand (antigen) against which activity is sought, i.e., a preselected antigen. The immunization can be carried out conventionally and antibody titer in the non-human animal can be monitored to determine the stage of immunization desired, which corresponds to the affinity or avidity desired. Partially immunized non-human animals typically receive only one immunization and cells are collected there from shortly after a response is detected. Fully immunized non-human animals display a peak titer that is achieved with one or more repeated injections of the antigen into the host non-human animal, normally at two to three week intervals.

Genes coding for V_{H} and V_{L} polypeptides can be derived from cells producing IgA, IgD, IgE, IgG or IgM. Methods for preparing fragments of genomic DNA from which immunoglobulin variable region genes can be cloned are well known in the art

As used herein, the term “specifically associate” or “specifically interact” or “specifically bind” refers to two or more polypeptides that form a complex that is relatively stable under physiologic conditions. The terms are used herein in reference to various interactions, including, for example, the interaction of a first polypeptide subunit and a second polypeptide subunit that interact to form a functional protein complex, as well as to the interaction of an antibody and its antigen. A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M, or 1×10⁻¹⁰ M or greater. A specific interaction generally is stable under physiological conditions, including, for example, conditions that occur in a cell or subcellular compartment of a living subject, which can be a vertebrate or invertebrate, as well as conditions that occur in a cell culture such as used for maintaining cells or tissues of an organism. Methods for determining whether two molecules interact specifically are well known and include, for example, equilibrium dialysis, surface plasmon resonance, gel shift analyses, and the like.

In one aspect, antibodies elicited in vivo can be evolved to enhance antibody affinity in vitro. For example, CDR walking can be used, where individual or multiple CDR regions of antibody heavy and light chains are sequentially randomized by saturation mutagenesis using overlapping PCR and NNK doping strategy. Libraries of Fab antibody sequence variants created in this way are displayed on phage surfaces and reselected against the antigen of interest (e.g., F-protein of HMPV) using a stringent and competitive panning environment to ensure the recovery of the highest affinity Fab clones. The affinity can then be determined by approaches known in the art (e.g., including, but not limited to, equilibrium dialysis, surface plasmon resonance, gel shift analyses, and the like). Using this method it is possible to enhance K_{d} values 10-, 100-, or 1000-fold.

In one embodiment, a panel of antibodies raised against HMPV F-protein is used to determine the neutralization properties of the antibodies against various forms of F-protein antigen. While not being bound by theory, conceptually, the F-protein could occur in multiple antigenic and immunogenic forms. It is not unreasonable that each of the immature and mature forms of F-protein are likely presented to the host immune system during natural infection and may elicit a different antibody profile, likely with some portion of overlapping cross reactivity deriving from antigenic determinants common to more than one of the various forms.

In a related aspect, the antigen binding properties of a panel of HMPV-specific antibodies are examined including neutralizing antibodies recovered by screening against a recombinant fusion of the HMPV F-protein that can be expressed in various systems including, but not limited to, a baculovirus system. In a further related aspect, other non-neutralizing Fabs obtained from various sources including, but not limited to, phage antibody libraries may comprise the panel. The non-neutralizing antibodies can be selected against a purified recombinant HMPV F-protein expressed in appropriate host cells (e.g., CHO cells). In one aspect, the antibodies exhibit neutralizing activity in vitro.

For example, subsequent to performing the above selection assay, competition studies using antibodies against HRSV (i.e., RSV19) indicated the non-neutralizing antibodies bound to epitopes other than that recognized by RSV19 antibody. More strikingly, the neutralizing antibody bound approximately 1000 times less well to the recombinant HRSV F-protein (K_{d}=6 nM) than the non-neutralizing antibodies. However, the efficiency with which RSV19 neutralized virus in vitro (50% plaque reduction at 4 nM) via a mechanism which must precede antibody binding to F-protein assembled into viron spikes, suggest that its overall binding constant for F-protein in this mature state is three orders of magnitude higher than for recombinant protein. Indeed, RSV19 bound much more efficiently than a non-neutralizing antibody panel to HRSV infected cell surfaces, where the protein is likely to be arranged into a mature, oligomeric structure ready for incorporation into the envelope of budding virions. Again, while not being bound by theory, these data support the hypothesis that this particular neutralizing antibody binds better to mature forms of F-protein than to immature forms, whereas the opposite binding pattern is seen with non-neutralizing antibodies.

In one embodiment, the HMPV F-specific Fabs of the present invention represent a diverse usage of V_{H} and V_{L} gene segments. In one aspect, the antibody variable antibody gene segments are segments that are not common in the repertoire of adult randomly selected B cells. For example, one or more neutralizing clones utilize distinct light chains but virtually identical heavy chains. While not being bound theory, this suggests that for such clones the heavy chain mediates the principal determinants for FΔTM binding. In another aspect, the highest in vitro neutralizing activity utilize distinct V_{H} and J_{H} segments at the nucleotide and amino acid level, where such Fabs may be derived from different donors. In a related aspect, the HCDR3 loop is the critical determinant antigen binding site.

Polynucleotides useful for practicing a method of the invention can be isolated from cells producing the antibodies of interest, for example, B cells from an immunized subject or from an individual exposed to a particular antigen, can be synthesized de novo using well known methods of polynucleotide synthesis, can be produced recombinantly or can be obtained, for example, by screening combinatorial libraries of polynucleotides that encode variable heavy chains and variable light chains. These and other methods of making polynucleotides encoding, for example, chimeric, humanized, CDR-grafted, single chain, and bifunctional antibodies are known to those skilled in the art.

The antibodies of the invention or fragments thereof can also be assayed for their ability to inhibit or downregulate HMPV replication using techniques known to those of skill in the art (see, e.g., U.S. Pat. No. 6,818,216, herein incorporated by reference). For example, HMPV replication can be assayed by plaque assay. The antibodies of the invention or fragments thereof can also be assayed for their ability to inhibit or downregulate the expression of HMPV polypeptides. Techniques known to those of skill in the art, including, but not limited to, ELISA, Western blot analysis, Northern blot analysis, and RT-PCR can be used to directly or indirectly measure the expression of HMPV polypeptides. Further, the antibodies of the invention or fragments thereof can be assayed for their ability to prevent the formation of syncytia.

The antibodies of the invention or fragments thereof are tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a subject tissue sample is grown in culture, and exposed to or otherwise administered an antibody or composition of the present invention, and the effect of such an antibody or composition of the present invention upon the tissue sample is observed. In various embodiments, in vitro assays can be carried out with representative cells of cell types involved in a HMPV infection (e.g., LLC-MK2 cells), to determine if an antibody or composition of the present invention has a desired effect against HMPV. In one aspect, the antibodies or compositions of the invention are also tested in in vitro assays and animal model systems prior to administration to humans. In a specific embodiment, cotton rats are administered an antibody the invention of fragment thereof, or a composition of the invention, challenged with 10⁵ pfu of HMPV, and four or more days later the rats are sacrificed and HMPV titer and anti-HMPV antibody serum titer is determined. Further, in accordance with this embodiment, the tissues (e.g., the lung tissues) from the sacrificed rats can be examined for histological changes.

In accordance with the invention, clinical trials with human subjects need not be performed in order to demonstrate the prophylactic and/or therapeutic efficacy of antibodies of the invention or fragments thereof. In vitro and animal model studies using the antibodies or fragments thereof can be extrapolated to humans and are sufficient for demonstrating the prophylactic and/or therapeutic utility of said antibodies or antibody fragments.

Antibodies or compositions of the present invention for use in therapy can be tested for their toxicity in suitable animal model systems including, but not limited to, rats, mice, cows, monkeys, and rabbits. For in vivo testing of an antibody or composition's toxicity any animal model system known in the art may be used.

Efficacy in treating or preventing viral infection may be demonstrated by detecting the ability of an antibody or composition of the invention to inhibit the replication of the virus, to inhibit transmission or prevent the virus from establishing itself in its host, to reduce the incidence of HMPV infection, or to prevent, ameliorate or alleviate one or more symptoms associated with HMPV infection. The treatment is considered therapeutic if there is, for example, a reduction is viral load, amelioration of one or more symptoms, a reduction in the duration of a HMPV infection, or a decrease in mortality and/or morbidity following administration of an antibody or composition of the invention. Further, the treatment is considered therapeutic if there is an increase in the immune response following the administration of one or more antibodies or fragments thereof which immunospecifically bind to one or more HMPV antigens.

Suitable labels for the antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷SC, ¹⁰⁹Pd, and the like.

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron.

Typical techniques for linking the above-described labels to antibodies include the use of glutaraldehyde, periodate, dimaleimide, m-maleimidobenzyl-N-hydroxy-succinimide ester, which are known in the art.

In one embodiment, a method of diagnosing a metapneumovirus (HMPV) infection is disclosed including contacting a sample from a subject with a human antibody that specifically binds to a HMPV fusion glycoprotein (F-protein) under conditions which allow for antibody/F-protein complex formation, contacting the sample with a reagent that interacts with the antibody/F-protein complex; and detecting the interaction of the reagent with the antibody, where detection of the reagent-antibody interaction is indicative of the presence of an HMPV infection in the subject. In one aspect, antibody/F-protein complex formation refers to an antibody-antigen interaction. In another aspect, reagent interaction includes, but is not limited to, binding of an antibody that recognizes the human antibody that specifically binds to the HMPV F-protein. Reagent interactions further include ligands that recognize moieties which are bound covalently or non-covalently to the antibody. For example, an antibody may be labeled with a biotin moiety, and the reagent would then comprise streptavidin. Other ligands useful for this purpose are known in the art, including the labels as outline above.

In another embodiment, a kit is disclosed comprising a device for contacting a biological sample with one or more human antibodies that specifically bind to one or more HMPV fusion glycoproteins (F-proteins) under conditions that allow for the formation of a complex between the one or more antibodies and one or more HMPV F-proteins, one or more reagents which remove non-complexed antibody, one or more reagents that recognize the antibody, instructions which provide procedures on the use of the antibody and reagents, and a container which houses the one or more antibodies, reagents, and instructions. In one aspect, a device can include, but is not limited to, a wick, a swab, porous media (e.g., beads, gels), a capillary tube, a syringe, a pipette, and the like. In a related aspect, the device may comprise a stationary phase where the sample serves as a mobile phase that is percolated through such a device.

In one aspect, different members of a diagnostic kit will depend on the actual diagnostic method to be used.

In addition to the above-listed possible members of the diagnostic kit, the kit may contain positive reference samples, negative reference samples, diluents, washing solutions, and buffers as appropriate.

In one aspect, diagnosis may comprise immunodiagnostic methods, such as enzyme-liked immunosorbent assay (ELISA), radioimmunoassay (RIA) or immunofluorescence assay (IFA).

For an ELISA, typically used enzymes linked to a polypeptide as a label include horseradish peroxidase, alkaline phosphatase, and the like. Each of these enzymes is used with a color-forming reagent or reagents (substrate) such as hydrogen peroxide and o-phenylenediamine; and p-nitrophenyl phosphate, respectively. Alternatively, biotin linked to a polypeptide can be utilized as a label to signal the presence of the immunoreactant in conjunction with avidin that is itself linked to a signaling means such as horseradish peroxidase.

In one aspect, F-protein may be detected by the method of the invention when present in biological fluids and tissues. Any specimen containing a detectable amount of such antigen can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis. In one aspect, the sample is blood, including serum. In a related aspect, the specimen is a human blood or serum sample.

The specific concentrations of the antibody and antigen, the temperature and time of incubation, as well as other assay conditions, can be varied, depending on such factors as the concentration of the antigen in the sample, the nature of the sample and the like. Those of skill in the art will be able to determine operative and optimal assay conditions for each determination by employing routine experimentation. Typically, the time period is predetermined for a given set of reaction conditions by well known methods prior to performing the assay.

Under biological assay conditions, the maintenance time period is usually from minutes to hours, such as 30 minutes to 2 hours to overnight, however, these time periods will vary. Other steps such as washing, stirring, shaking, filtering, or pre-assay extraction of antigen, and the like, may, of course be added to the assay, as may be desired or necessary for a particular situation. The complex formed can then be detected by means described herein.

All of the above mentioned diagnostic methods are well known in the art, and one of ordinary skill in the art will readily select useful members for a diagnostic kit in relation to the diagnostic method to be used.

A composition of the invention can be formulated such that it is in a form suitable for administration to a living subject, for example, a vertebrate or other mammal, which can be a domesticated animal or a pet, or can be a human. For example, a suitable form can be a composition comprising an encoded antibody, or antigen binding fragment thereof, the composition can be useful for passive immunization of a subject such as an individual exposed to a HMPV. As such, the present invention provides a medicament useful for ameliorating a pathologic condition such as a respiratory infection caused or exacerbated by HMPV.

In one embodiment, passive immunization allows for the delivery to a subject an anti-viral antibody at a consistently protective concentration, rather than relying on the vagaries of a natural immune response that would be encountered when vaccinating against HMPV. In a related aspect, a single administration of a small number of different antibodies of different specificities, simultaneously, protect the subject from infection by several viral respiratory pathogens.

In one embodiment, a vaccine is disclosed, including one or more human antibodies that specifically bind to a human metapneumovirus (HMPV) fusion glycoprotein (F-protein).

In one aspect, the vaccine may additionally comprises an adjuvant. Such an adjuvant must of course be an adjuvant which is approved for use in vaccines by authorities responsible for veterinary or human medicines.

Individual antibodies or vaccine preparations containing antibodies can be incorporated into compositions for diagnostic or therapeutic use. The form depends on the intended mode of administration and diagnostic or therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. See Remington's Pharmaceutical Science, (15th ed., Mack Publishing Company, Easton, Pa., 1980). Compositions intended for in vivo use are usually sterile. Compositions for parental administration are sterile, substantially isotonic, and made under GMP condition.

The formulation is administered to a mammal in need of treatment with the antibody, including a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In one embodiment, the formulation is administered to the mammal by intravenous administration. For such purposes, the formulation may be injected using a syringe or via an IV line, for example.

The appropriate dosage (“therapeutically effective amount”) of the antibody will depend, for example, on the condition to be treated, the severity and course of the condition, whether the antibody is administered for preventive or therapeutic purposes, previous therapy, the patient's clinical history and response to the antibody, the type of antibody used, and the discretion of the attending physician. The antibody is suitably administered to the patient at one time or over a series of treatments and may be administered to the patient by any time from diagnosis onwards. The antibody may be administered as the sole treatment or in conjunction with other drugs or therapies useful in treating the condition in question.

As a general proposition, the therapeutically effective amount of the antibody administered will be in the range of about 0.1 to about 50 mg/kg of patient body weight whether by one or more administrations, with the typical range of antibody used being about 0.3 to about 20 mg/kg, and/or about 0.3 to about 15 mg/kg, administered daily, for example. However, other dosage regimens may be useful. The progress of this therapy is easily monitored by conventional techniques.

The following examples are intended to illustrate, but not limit, the scope of the invention.

### EXAMPLES

### Materials and Methods

HMPV F Ectodomain Expression in Mammalian Cells.

RT-PCR was used to amplify a full length F sequence from a pathogenic clinical isolated designated TN/92-4, a prototype genogroup A2 strain according to the proposed nomenclature (van den Hoogen et al., Nat Med (2001) 7:719-724). The full TN/92-4 F sequence was sequence-optimized by commercial source (Aptgen) to alter suboptimal codon usage for mammalian tRNA bias, improve secondary mRNA structure and remove AT-rich regions, increasing mRNA stability. An expression vector was then generated encoding the HMPV F ectodomain construct lacking transmembrane (TM) domain (pcDNA3.1-FΔTM). The optimized full-length cDNA of the F gene was PCR amplified with primers 5′-GGAGGTACCATGAGCTGGAAG-3′ and 5′-GAAGCGGCCGCTGCCCTTCTC-3′ and PCR product was digested and ligated into the KpnI/NotI sites (restriction sited underlined in the primer sequences) of the vector pcDNA3.1/myc-His B (Invitrogen). The pcDNA3.1-FΔTM recombinant plasmid was transfected into suspension 293-F cells (Freestyle 293 Expression System, Invitrogen). At 96 hours post-transfection, cells were centrifuged for 5 min at 100×g at room temperature and supernatant harvested. Supernatant was filtered through 0.2 μm filters before purification.

Purification of 6×His-tagged F Ectodomain.

Protein purification was performed on a ÄKTA FPLC system controlled by UNICORN 4.12 software (GE Healthcare). The His-tagged F ectodomain FΔTM was purified by immobilized metal ion chromatography using pre-packed HisTrap Ni-Sepharose columns (GE Healthcare). Sample was diluted with concentrated binding buffer stock to adjust pH, salt and imidazole concentration before purification. Protein was loaded on a 5 ml HisTrap column with a loading flow rate of 5.9 ml/min, and the binding buffer contained 20 mM sodium phosphate, 0.5 M NaCl, 30 mM imidazole (pH 7.4). Unrelated proteins were eluted in elution step 1 using 4 column volumes of 8% elution buffer and the 6×His-tagged F protein was eluted in elution step 2 with 4 column volumes of 25% elution buffer. The elution buffer contained in 20 mM sodium phosphate, 0.5 M NaCl, 500 mM imidazole (pH 7.4). Purified protein was concentrated and dialyzed against PBS (Invitrogen) through Amicon Ultra centrifugal filters with 30,000 and 100,000 molecular weight cut off (MWCO, Millipore).

Construction and Selection of Antibody Phage Display Libraries.

Antibody Fab immunoglobulin G1 (IgG1) (K and or X chain) phage display libraries were cloned from the bone marrow tissue of 12 donors as described (Barbas et al., Proc Natl Acad Sci USA (1992) 89:10164-10168, Williamson et al., Proc Natl Acad Sci USA (1993) 90:4141-4145). Libraries ranged in size from 3×10³ to 5×10⁷ members. Libraries were selected individually against recombinant HMPV F protein bound to enzyme-linked immunosorbant assay (ELISA) wells using a biopanning procedure as described (Barbas et al. (1992)). Selected phage recovered from the fourth or fifth rounds of panning were converted to a soluble Fab expression system (Barbas et al. (1992)), and clones were tested individually for reactivity with the recombinant HMPV F protein selecting antigen. Selected HMPV F protein-reactive Fab clones were purified by immuno-affinity chromatography (Williamson et al. (1993)). The light-chain and heavy-chain variable region sequences of HMPV-reactive antibody Fab clones were determined as described (Williamson et al. (1993)). V_{H} or V_{L} regions sequences were analyzed with international ImMunoGeneTics database (hosted by Centre Informatique National de L'Enseignment Supérieur, Montpellier, France) using the junction analysis program, reporting results with an updated nomenclature of the human Ig genes as recently summarized (Giudicelli et al., Nucleic Acid Res (2006) 34:D781-784, Ruiz et al., Nucleic Acids Res (2000) 28:219-221). All V_{H} and V_{L} assignments were reviewed and confirmed by manual inspection. Mutations in the junction region were manually confirmed, and mutations in the remaining regions were manually scored and tabulated.

Immunofluorescent Assay.

LLC-MK2 cell culture monolayers were infected with HMPV at an MOI of 1. At 34 h after infection, cells were fixed with 10% buffered formalin, washed with PBS-T then incubated with either Fabs or anti-HMPV serum (diluted 1:500) in PBS-T/milk for 1 h at 37° C. After washing with PBS-T, cells were stained with AlexaFluor586-conjugated goat anti-guinea pig Ig or AlexaFluor568-conjugated mouse anti-Fab antibody diluted 1:1000 (Molecular Probes) in PBS-T/milk for 1 h at 37° C. Cell monolayers were examined on an inverted Nikon Diaphot microscope and images captured with a Nikon D100 digital camera. Images were cropped and figures constructed using Adobe Photoshop and Illustrator without digital adjusting or reprocessing of images.

In Vitro Neutralization Assays.

HMPV-neutralizing titers were determined by a plaque reduction assay as described (Williams et al., J Virol (2005) 79:10944-10951), with the following modifications. Fab suspensions in serial 4-fold dilutions, starting with undiluted, were incubated with a working stock of HMPV diluted to yield 50 plaques per well in a 24-well plate. The Fab and virus mixture was incubated for 1 h at 37° C. with rotation. The Fab/virus mixtures then were plated in triplicate on LLC-MK2 monolayers in 24-well culture plates and allowed to adsorb at room temperature for 1 h. Wells were then overlaid with 0.755 methylcellulose in OptiMEM supplemented with trypsin and incubated at 37° C. in a CO₂ incubator for four days. Monolayers were rinsed, formalin fixed and stained with guinea pig anti-HMPV serum and peroxidase-labeled goat anti-guinea pig Ig as described (Williamson et al. (2005)). Plaques were counted and 60% plaque reduction titers were calculated. HMPV positive human serum was used as a positive control.

Surface Plasmon Resonance.

The interaction of HMPV F-specific MAbs with HMPV F protein was performed using surface Plasmon resonance on a BIAcore 2000. Purified recombinant HMPV F or RSV F protein were diluted to 30 μg/ml in 10 mM sodium acetate, pH 4.5, and covalently immobilized at 45° l/ml by amine coupling to the dextran matrix of a CM5 sensor chip (BIAcore Life Sciences) with a target RU density of 1200. Unreacted active ester groups were blocked with 1 M ethanolamine. For use as a reference, a blank surface, containing no protein, was prepared under identical immobilization conditions. Purified HMPV F antibodies and RSV-specific MAb (palivizumab), at different concentrations ranging from 5 to 500 nM in HBS/Tween-20 buffer (BIAcore Life Sciences), were injected over the immobilized HMPV F protein, RSV F protein, to reference cell surfaces. Antibody binding was measured at a flow rate of 30 μl/min for 180 seconds and dissociation was monitored for an additional 360 seconds. Residual bound antibody was removed from the sensor chip by pulsing 50 mM HCl at 100 μl/min for 30 seconds. Kₐ, K_{d}, and KD were determined by aligning the binding curves globally to fit a 1:1 Langmuir binding model using BIAevaluation software.

In Vivo Infection and Fab Treatment.

Cotton rats were purchased at 5-6 weeks of age from a commercial breeder (Harlan, Indianapolis, Ind.), fed standard diet and water ad libitum and kept in microisolator cages. Animals were anesthetized by isofurane inhalation prior to virus or Fab inoculation. The virus strain used was a pathogenic clinical isolate designated hMPV strain TN/94-49, a genotype group A2 virus, according to the proposed nomenclature (van den Hoogen et al. (2004)). This virus stock was determined to have a titer of 3.5×10⁶ pfu/ml by plaque titration in LLC-MK2 cell monolayer cultures. Cotton rats in groups of 5-1 were inoculated on day 0 intranasally with 3.5×10⁵ pfu in a volume of 100 μl. On day 3 post-infection, solutions of Fab were instilled intransally. An irrelevant similarly prepared Fab designated B12 was used at 1 or 4 mg/kg body weight. The HMPV F-specific DSλ7 was used at 0.06, 0.25, 1 or 4 mg/kg body weight. All Fab concentrations were adjusted to a uniform volume of 100 μl except for the B12 4 mg/kg dose, which was given in a 225 μl volume due to lower concentration. On day 4 post-infection (24 hours after the Fab administration), the animals were sacrificed by CO₂ asphyxiation, and exsanguinated. Nasal and lung tissues were harvested separately, weighed individually for each animal and homogenized immediately. The lungs were pulverized in ice cold glass homogenizers and nasal turbinates were ground with sterile sand in a cold porcelain mortar ad pestle in 3 ml of ice-cold Hank's balanced salt solution. Tissue homogenates were centrifuged at 4° C. for 10 minutes at 300×g and the supernatants were collected, aliquoted into cryovials and snap-frozen in liquid nitrogen. The Vanderbilt Institutional Animal Care and Use Committee approved the study.

Statistical Analysis.

Viral titers between control groups were compared with the Krustal-Wallis test. Viral titers in each of the HMPV F-specific Fab DS27-treated groups were compared with the viral loads in the combined control groups using a Wilcoxon rank sum test. Linear regression was used to examine a dose-response analysis. The doses were log₂ transformed, since the doses 2⁻⁴, 2⁻², 2⁰, and 2² mg/kg, and tissue virus titers were log-transformed to minimize the effect of a non-Gaussian distribution. Viral assays in which plaques were not detected were assigned a titer at the detection limit of 5 PFU/g before log₁₀-transformation. In this model, a line was fitted to the data, since it was reasoned that with only 4 distant dose levels, models that fit flexible curves to the data could be over-fitting the data. Titers of experimental groups were expressed as geometric mean titer.

Recovery of HMPV F-specific monoclonal Fab fragments by phage library panning. Phage antibody Fab display libraries prepared from bone marrow tissues of 12 donors were selected individually against recombinant HMPV F protein bound to ELISA wells. Twenty or thirty antibody Fab clones present after the fourth or fifth round of phage panning were evaluated in an ELISA for reactivity against the selecting antigen. Antigen-specific clones were isolated from 5 of the 12 donor libraries. Analysis of the Fab light-chain and heavy chain DNA sequences of the specific Fabs identified 14 different clones with distinct sequences (Table 1).

Selection experiments for HMPV were performed against affinity purified F-protein in which the transmembrane portion of the molecule has been deleted (ATM) (Provided by John Williams, Vanderbilt University). The selection experiments resulted in a panel of 14 different HMPV-reactive antibodies, as determined by heavy chain sequencing, as shown in Table 1.

In these experiments, only one of the 6 phage antibodies failed to yield HMPV-specific antibodies after a single panning. As soluble Fab fragments, each of the antibodies listed in Table 1 were shown to react positively with recombinant F-protein in an ELISA format, but not with control antigens.

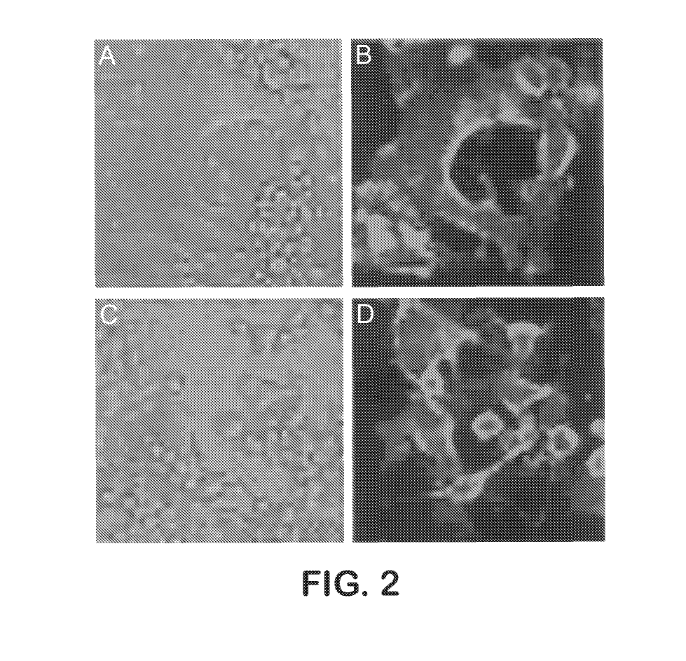

To further investigate their reactivity, the ELISA-positive Fab antibodies were evaluated in an immunohistochemical assay against HMPV-infected LLC-MK2 cells Bacterial supernatants from 14 Fab clones that specifically bound HMPV F-protein by ELISA screening were tested. Of the 14 Fab antibodies tested, all except two exhibited specific binding to HMPV-infected cells (FIG. 2A,B). Several Fabs exhibited neutralizing activity in vitro and were purified from bacterial supernatants. These purified Fabs also bound to HMPV-infected cells (FIG. 2C,D). The F-specific Fabs detected both syncytia and single infected cells in a membrane-distributed pattern consistent with the expected localization of F protein. The pattern of fluorescence was similar to that seen previously with staining of HMPV-infected cells with polyclonal serum, or cells transfected with cDNA encoding HMPV F alone. Fab clones that detected HMPV by immunofluorescence were tested further in vitro neutralizing ability.

To determine the functionality of the HMPV-specific Fabs, a microneutralization plaque assay was employed. Initially, crude bacterial supernatants containing soluble F-protein reactive Fab clones were screened. The dilution of bacterial supernatant required to achieve 60% reduction in the plaque count was recorded in each case. A recombinant Fab recognizing HIV-1 and human serum with high HMPV neutralization titer were incorporated in this experiment as negative and positive controls, respectively. These experiments indicated that some 4 individual Fab clones, DSλ1, DSλ6, DSλ7, and ACN044 possessed HMPV neutralization activity against the A2 strain of HMPV. Each of these antibodies, together with the non-neutralizing HMPV F-protein specific antibody Hanκ9, and Fab b12, were then purified by affinity chromatography and the HMPV neutralization experiments repeated. The results of these experiments are shown in Table 2.

TABLE 2
Neutralization of HMPV (A2 strain) by purified F-
protein specific recombinant human antibody Fabs.

| | | | | Neut. Conc. |
|---|---|---|---|---|
| | | | Neut. Dilution | (60% plaque |
| | | Conc. of Fab | (60% plaque | reduction) |
| Fab | Code | (μg/ml) | reduction) | (μg/ml) |
| DSλ1 | A | 160 | 1:65 | 2.5 |
| DSλ6 | B | 178 | 1:55 | 3.2 |
| DSλ7 | C | 1180 | 1:1114 | 1.1 |
| Hanκ9 | D | 66 | <1:20 | N.A. |
| ACN044 | E | 191 | 1:144 | 1.3 |
| b12 | F | 1500 | <1:20 | N.A. |

The data indicate that Fab clones DSλ1, DSλ6, DSλ7, and ACN044 neutralize the A2 strain of HMPV in vitro with reasonable efficiency (1.1 to 3.2 μg/ml). Subsequently, it was determined whether or not the Fab panel could also neutralize strains of HMPV other than A2 (Table 3). Although A2 is thought to be the dominant HMPV genogroup in the clinic, other genogroups, and particularly B2, may also be commonly encountered.

TABLE 3
Neutralization activity (60% plaque reduction) of selected human
Fabs against HMPV strains from all four known genogroups.

| | | | | Neut. | | Neut. | | Neut. | | Neut. |
|---|---|---|---|---|---|---|---|---|---|---|
| | | Conc. | Neut. | Conc. | Neut. | Conc. | Neut. | Conc. | Neut. | Conc. |
| Fab | | of Fab | Dilution | (μg/ml) | Dilution | (μg/ml) | Dilution | (μg/ml) | Dilution | (μg/ml) |
| Name | Code | (μg/ml) | A1 | | A2 | | B1 | | B2 | |
| DSλ1 | A | 160 | <1:20 | N.A. | 1:39 | 4.1 | <1:20 | N.A. | <1:20 | N.A. |
| DSλ6 | B | 178 | <1:20 | N.A. | 1:84 | 2.1 | <1:20 | N.A. | <1:20 | N.A. |
| DSλ7 | C | 1180 | 1:120 | 9.8 | 1:1042 | 1.1 | 1:488 | 2.4 | <1:20 | N.A. |
| ACN044 | E | 191 | <1:20 | N.A. | 1:86 | 2.2 | <1:20 | N.A. | <1:20 | N.A. |

The data confirmed that all of the selected antibodies neutralized HMPV A2 genogroup, however only the DSλ7 Fab clone displayed any neutralization activity against the A1 or B1 genogroups. These data suggest that the antibodies recovered by selecting against recombinant F-protein representative of the A2 genogroup favored selection of antibodies neutralizing A2 virus. This type of genogroup-specific neutralization is unexpected given the high amino acid identity between F-proteins associated with the different HMPV genogroups, and adds weight to the argument that selective pressure exerted by a neutralizing antibody response can play an important role in shaping envelope protein heterogeneity within the Paramyxoviridae family.

Below is a comprehensive comparison of F-protein sequences across and within the 4 HMPV genogroups as encountered in archived clinical samples (Table 4), as exemplified by SEQ ID NO:30 (A1); SEQ ID NO:32 (A2); SEQ ID NO:34 (B1) and SEQ ID NO:36 (B2).

TABLE 4
Comparison of nucleotide and amino acid identity of full-length
human metapneumovirus F genes within and between genogroups.

| | Minimum % | Mean % | Minimum % | Mean % |
|---|---|---|---|---|
| | nucleotide | nucleotide | amino acid | amino acid |
| Group (n) | identity | identity | identity | identity |
| A1 (10) | 97.5 | 98.2 | 99.3 | 99.6 |
| A2 (24) | 97.2 | 98.7 | 98.9 | 99.6 |
| B1 (9) | 97.6 | 98.5 | 98.7 | 99.3 |
| B2 (35) | 93.5 | 97.5 | 99.4 | 99.9 |
| A1 vs. A2 (34) | 93.9 | 96 | 98 | 98.7 |
| A1 vs. B1 (19) | 84 | 91.3 | 93.7 | 97 |
| A1 vs. B2 (45) | 83.7 | 86.7 | 94.2 | 95.7 |
| A2 vs. B1 (33) | 84 | 94.7 | 93.9 | 98.1 |
| A2 vs. B2 (59) | 84.1 | 89.7 | 94.6 | 96.7 |
| ALL (78) | 83.7 | 89 | 93.7 | 96.3 |

The HMPV FΔTM-specific Fabs utilized a number of VH gene segments (Table 5).

TABLE 5
Heavy-chain and light-chain variable region segment
usage of recombinant human antibody Fabs reacting
specifically with recombinant HMPV F protein.

| Fab | VH | D | JH | VL | JL |
|---|---|---|---|---|---|
| AC31 | 1-03 | 3-10 | JH3 | K3-20 | JK1 |
| AC59 | 1-02 | 6-13 | JH3 | K1-39 | JK3 |
| AC69 | 3-23 | 6-19 | JH5 | L6-57 | JL3 |
| AC83 | 4-39 | 3-22 | JH3 | K1-39 | JK1 |
| ACN044 | 4-59 | 1-26 | JH3 | L3-01 | JL2/3 |
| DS1 | 4-59 | NA | JH5 | L1-40 | JL2/3 |
| DS6 | 4-59 | NA | JH5 | L2-14 | JL/23 |
| DSλ7 | 3-66 | 1-26 | JH3 | L3-01 | JL2/3 |
| Han01 | 1-03 | 3-08 | JH4 | K1-NL1 | JK4 |
| Han02 | 3-49 | NA | JH6 | L2-23 | JL2/3 |
| Han05 | 1-03 | 3-09 | JH4 | K3-20 | JK1 |
| Han09 | 3-11 | NA | JH3 | K2-30 | JK2 |
| Han10 | 3-49 | NA | JH6 | L1-51 | JL3 |
| LL01 | 1-03 | 3-09 | JH4 | K1-NL1 | JK4 |

Bold print indicates Fab clones with in vitro neutralizing activity. Clones derived from distinct donor libraries: all designated “AC;” all designated “DS;” Han01 and Han05; Han02; Han09; and Han10; LL01.

VH3-23 was only present in one clone, despite being the most commonly used V_{H} segment in the adult random circulating B cell repertoire (Brezinchek et al., J Immunol (1995) 155:190-202, Corbett et al., J Mol Biol (1997) 270:587-597, Weitkamp et al., J Immunol (2003) 171:4580-4688). V_{H}1-03, which is utilized by fewer than 5% of random circulating B cells, was used by four clones. V_{H} 4-59 was utilized in three clones, two that were likely clonally related from one donor, and one from a separate donor. Of the four Fab clones with virus neutralizing activity, two from a single donor (DSλ1 and DSλ6) had very similar V_{H} segments and identical HCDR3 regions (Table 1), but distinct light chains. The two clones with the highest neutralizing ability (ACN044 and DSλ7) comprised V_{H}, J_{H} and light chains that were distinct at the nucleotide and amino acid level, but had very similar HCDR3 loops (Table 1). Analysis of somatic mutations revealed that most of the Fab clones were highly mutated, with framework mutations predominant (Table 6A and 6B).

However, there was no apparent correlation between the number of mutations and neutralizing activity.

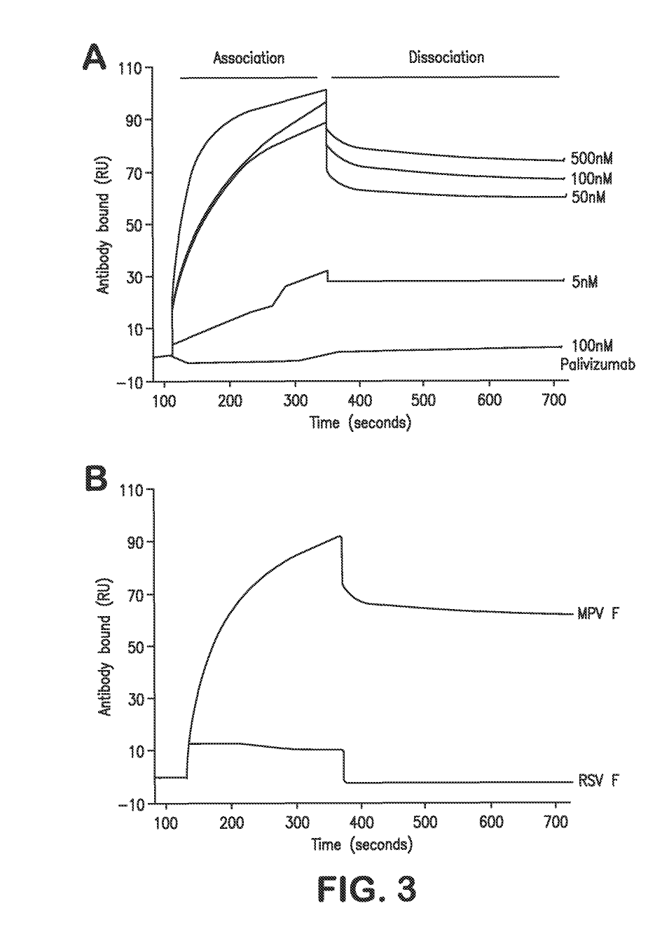

SPR studies indicated that HMPV-specific Fab bound HMPV FΔTM with high affinity, while as expected, the RSV F-specific MAb palivizumab did not. The binding curves of anti-HMPV Fab DSλ7 at concentration ranging from 500 nM to 5 NM showed a pattern of specific binding to HMPV FΔTM (FIG. 3). In contrast, FIG. 3 shows that palivizumab did not bind to HMPVFΔTM even at 100 nM concentration. We tested the binding ability of palivizumab to RSV-FΔTM, and it exhibited strong, specific binding, showing that the lack of binding to HMPVFΔTM was the result of specificity and not related to the quality of the antibody. The affinity of the human Fab DSλ7 for HMPVFΔTM was high, with Kₐ=3.54×105 (1/ms), K_{d}=3.48×10⁻⁵ (1/s) and K_{D}=9.84×10⁻¹⁰ (M). These values suggest a strong, specific antibody-antigen binding. The binding Fab DSλ7 showed specific binding to HMPVFΔTM, but did not have a detectable affinity for RSV FΔTM protein (FIG. 3).

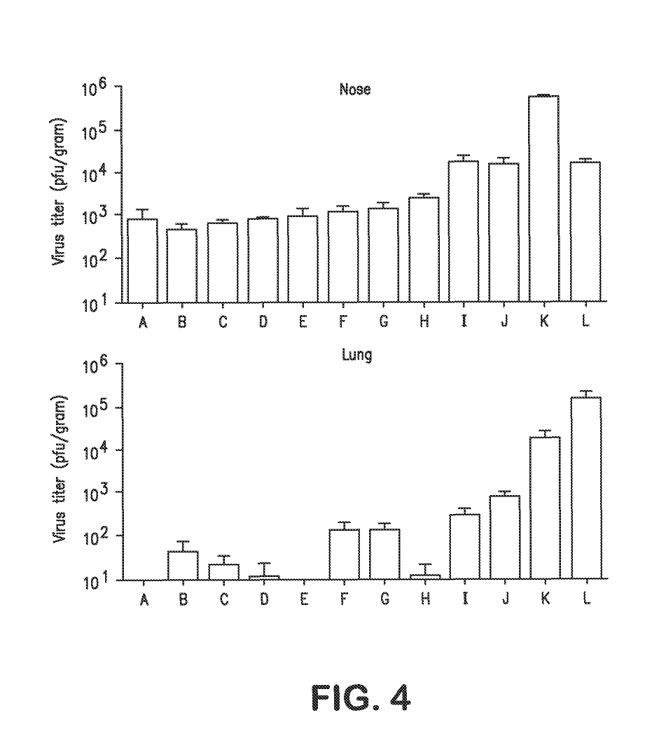

Hamsters, guinea pigs, cotton rats, and nine inbred strains of mice were inoculated intranasally with 10⁵ pfu of HMPV under anesthesia. The animals were sacrificed 4 days post-infection and HMPV titer in nose and lung tissues determined by plaque titration. None of the animals exhibited respiratory symptoms, which is common in rodent models of paramyxovirus infection. Studies of RSV infection in mice have shown that the mice exhibit symptoms such as huddling and ruffled fur only with a very high inoculum of 10⁸ pfu. The quantity of virus present in nasal tissue ranged from 4.6×10² pfu/g tissue (C3H mice) to greater than 10⁵ pfu/g tissue (hamster) (FIG. 4, top). Thus all animals were semi-permissive for HMPV replication in nasal turbinates. Determination of lung titers yielded quite different results (FIG. 4, bottom). The amount of HMPV replicating in lung tissue ranged from less than detectable (<5 pfu/g; guinea pigs and SJL mice) to a mean of 1.8×10⁵ pfu/g (cotton rat).

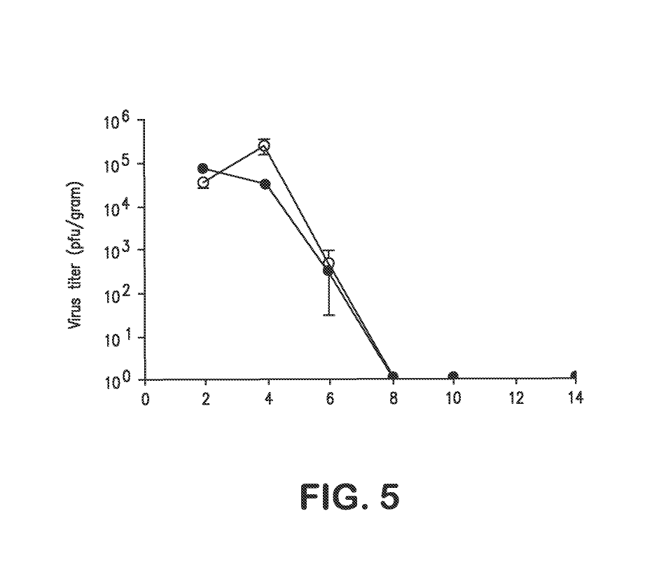

These data indicate that among the rodents tested, the cotton rat was the most highly permissive for HMPV infection. Thus, the kinetics of HMPV replication in cotton rats was determined by infecting animals intranasally with 10⁵ pfu of HMPV and sacrificing them at 2, 4, 6, 8, 10, or 14 days post-infection. As shown in FIG. 5, HMPV replication peaked in the nasal turbinates on day 2 at a mean titer of 5.6×10⁴ pfu/g, declined after day 4, and was not detected in nasal turbinates after day 6. The replication of HMPV in the lung tissues peaked on day 4 post-infection at a mean titer of 1.8×10⁵ pfu/g and declined rapidly, with virus undetected in the lung after day 6. This is similar to data on duration of HMPV shedding in humans (Ebihara et al., J Clin Microbiol (2004) 42:126-132; van den Hoogen et al., J Infect Dis (2003) 188:1571-1577; Williams et al., J Infect Dis (2006) 193:387-395).

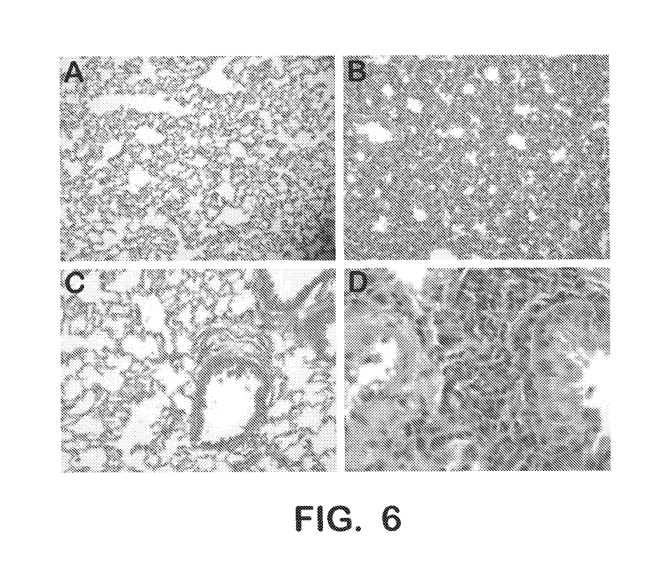

Next, pathologic specimens from uninfected and infected animals harvested 4 days post-infection were examined. The nasal epithelium of the HMPV-infected animals showed mild subepithelial lymphoid infiltrates. The most striking findings were in the lungs of infected animals (FIG. 6B,D). The lungs of HMPV-infected cotton rats showed peribronchial lymphoplasmocytic infiltrates and edematous thickening of the bronchial submucosa. In addition, there was diffuse mild expansion of the alveolar interstitium due to mononuclear cell infiltrates and edema. Sloughed epithelial cells, neutrophils, macrophages, and amorphous debris were visible in the bronchial lumens. The distribution of the lung lesions was multifocal and they were locally extensive. The lungs of the uninfected animals were normal (FIG. 6A,C). Pathological changes were not seen in sections of the brain, heart, thymus, lung spleen, or liver in any animals.

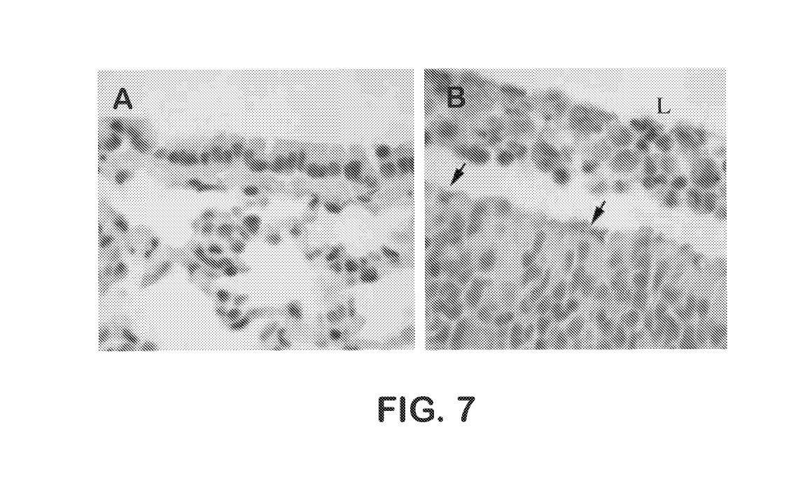

Tissue sections, including lung, from the uninfected animal did not exhibit reactivity with the anti-HMPV serum (FIG. 7A). Immunostained sections of brain, heart, thymus, spleen, and liver from HMPV-infected cotton rats were negative. HMPV antigen was only detected in respiratory epithelial tissue in sections from HMPV-infected cotton rats, at the luminal surface of respiratory epithelial cells (FIG. 7B). HMPV antigen staining was seen in respiratory epithelial cells from nasal tissue to the bronchioles in both morphologically normal and degenerated epithelial cells, indicating viral replication in the respiratory epithelium. Luminal cellular debris that included both sloughed epithelial cells and macrophages stained positive for HMPV antigen. Immunohistochemistry for CD3 showed a substantial influx of T cells, suggesting that cellular immunity participated in clearance of virus. Both histopathology and immunohistochemistry data reflected human and primate studies that have failed to identify HMPV in tissues other than the respiratory tract.

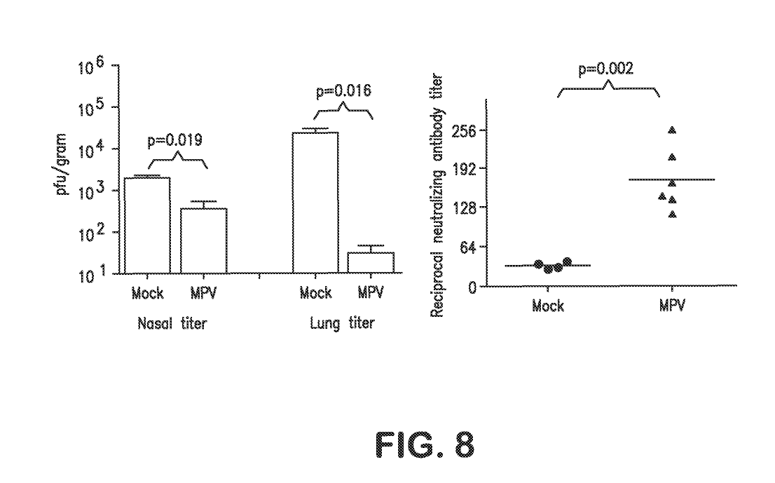

To determine protective immunity and antibody responses, two groups of cotton rats were inoculated intranasally with either sucrose (mock) or 10⁵ pfu of sucrose purified HMPV. Both groups were challenged 21 days later with 10⁵ pfu of sucrose purified HMPV intranasally. Four days later cotton rats were sacrificed and virus titers determined in nasal and lung tissues. The previously infected animals exhibited a modest but significant reduction in nasal titer, and showed a high degree of protection in the lungs (FIG. 8, left). Four of the six previously infected rats did not shed detectable titers of virus in the lungs. The absence of viral replication in these animals was confirmed by immunohistochemistry. The previously infected animals also mounted significant serum neutralizing antibody titers, with a mean of 1:174 (range 1:117-1:256) (FIG. 8, right).

These data show that cotton rats have proven permissive for HMPV replication in the nose and lungs, where pathology is consistent with bronchoilitis. Moreover, HMPV infection induces protective immunity in the cotton rat. The cotton rat thus provides a robust and tractable animal model of HMPV infection.

To determine the functionality of the HMPV-specific Fabs in vivo, seven groups of cotton rats (6 to 7 animals/group, total of 43 animals) were selected. Four groups were administered intranasally with Fab DSλ7 at 0.06 mg/kg, 0.25 mg/kg, 1 mg/kg, and 4 mg/kg. As a control, two groups were administered intranasally with Fab b12 (non-neutralizing antibody) at 4 mg/kg and 1 mg/kg, respectively. The following day, the animals were inoculated intranasally with 10⁵ pfu of sucrose purified HMPV. After 4 days, the animals were sacrificed and virus titers determined in nasal and lung tissues. Each set of test and control animals was compared to a group of animals receiving only HMPV. The results are shown in Table 7.

TABLE 7
Reduction in viral load by administration of human Fab DSλ7 in HMPV infected Cotton rats.

| | | | | | | T test | | | | | |
|---|---|---|---|---|---|---|---|---|---|---|---|
| | | Lung | | | Mean | v. HMPV | Nose | | | Mean | T test v. |
| Group | Rat # | Weight | Pfu/ml | Pfu/gm | Titer | (P value) | Weight | Pfu/ml | Pfu/g | Titer | HMPV |
| b12 | 1 | 0.45 | 1700 | 1.13E+04 | | | 1.71 | 7030 | 12333.33 | | |
| 4 mg/kg | 2 | 0.45 | 1130 | 7.53E+03 | | | 1.78 | 12000 | 20224.72 | | |
| | 3 | 0.44 | 2370 | 1.62E+04 | | | 2.53 | 8000 | 9486.17 | | |
| | 4 | 0.55 | 86 | 4.69E+02 | | | 2.29 | 5600 | 7336.24 | | |
| | 5 | 0.51 | 3700 | 2.18E+04 | | | 2.23 | 11300 | 15201.79 | | |
| | 6 | 0.46 | 0 | 0.00E+00 | 9543.26 | 0.740 | 2.35 | 6300 | 8042.55 | 12104.13 | 0.010 |
| b12 | 7 | 0.46 | 5300 | 3.46E+04 | | | 1.34 | 2300 | 5149.25 | | |
| 1 mg/kg | 8 | 0.43 | 1730 | 1.21E+04 | | | 1.67 | 11700 | 21017.96 | | |
| | 9 | 0.47 | 1900 | 1.21E+04 | | | 1.8 | 13700 | 22833.33 | | |
| | 10 | 0.36 | 2500 | 2.08E+04 | | | 2.28 | 43300 | 56973.68 | | |
| | 11 | 0.48 | 1270 | 7.94E+03 | | | 1.99 | 12700 | 19145.73 | | |
| | 12 | 0.36 | 1800 | 1.50E+04 | 17088.91 | 0.091 | 1.5 | 4300 | 8600.00 | 22286.66 | 0.199 |
| HMPV | 13 | 0.53 | 1200 | 6.79E+03 | | | 1.37 | 11300 | 24744.53 | | |
| | 14 | 0.44 | 900 | 6.14E+03 | | | 1.68 | 20000 | 35714.29 | | |
| | 15 | 0.36 | 2030 | 1.69E+04 | | | 1.81 | 14000 | 23204.42 | | |
| | 16 | 0.49 | 0 | 0.00E+00 | | | 1.89 | 40000 | 63492.06 | | |
| | 17 | 0.48 | 400 | 2.50E+03 | | | 1.64 | 15700 | 28719.51 | | |
| | 18 | 0.46 | 2400 | 1.57E+04 | 7999.61 | NA | 1.96 | 24700 | 37806.12 | 35613.49 | NA |
| DSλ7 | 19 | 0.47 | 100 | 6.38E+02 | | | 1.71 | 9700 | 17017.54 | | |
| 0.6 mg/kg | 20 | 0.53 | 70 | 3.96E+02 | | | 1.77 | 9000 | 15254.24 | | |
| | 21 | 0.5 | 0 | 0.00E+00 | | | 1.89 | 9700 | 15396.83 | | |
| | 22 | 0.5 | 213 | 1.28E+03 | | | 1.75 | 14000 | 24000.00 | | |
| | 23 | 0.48 | 7 | 4.38E+01 | | | 1.6 | 24300 | 45562.50 | | |
| | 24 | 0.47 | 3.3 | 2.11E+01 | 396.22 | 0.042 | 1.92 | 10300 | 16093.75 | 22220.81 | 0.117 |
| DSλ7 | 25 | 0.46 | 7 | 4.57E+01 | | | 1.93 | 8700 | 13523.32 | | |
| 0.25 mg/kg | 26 | 0.51 | 10 | 5.88E+01 | | | 1.75 | 13700 | 23485.71 | | |
| | 27 | 0.48 | 20 | 1.25E+02 | | | 1.47 | 18300 | 37346.94 | | |
| | 28 | 0.41 | 23 | 1.68E+02 | | | 1.75 | 8000 | 13724.29 | | |
| | 29 | 0.42 | 10 | 7.14E+01 | | | 1.5 | 2900 | 5800.00 | | |
| | 30 | 0.39 | 83 | 6.38E+02 | 184.61 | 0.039 | 1.57 | 9700 | 18535.03 | 18734.21 | 0.051 |
| DSλ7 | 31 | 0.46 | 30 | 1.96E+02 | | | 1.34 | 12700 | 28432.84 | | |
| 1 mg/kg | 32 | 0.43 | 100 | 6.98E+02 | | | 1.92 | 8300 | 12968.75 | | |
| | 33 | 0.45 | 13 | 8.67E+01 | | | 1.81 | 6700 | 11104.97 | | |
| | 34 | 0.5 | 100 | 6.00E+02 | | | 1.67 | 3500 | 6287.43 | | |
| | 35 | 0.38 | 10 | 7.89E+01 | | | 1.75 | 9000 | 15428.57 | | |
| | 36 | 0.4 | 100 | 7.50E+02 | 401.49 | 0.043 | 1.74 | 4700 | 8103.45 | 13721.00 | 0.014 |
| DSλ7 | 37 | 0.47 | 0 | 0.00E+00 | | | 1.52 | 9700 | 19144.74 | | |
| 4 mg/kg | 38 | 0.43 | 0 | 0.00E+00 | | | 1.71 | 3000 | 5263.16 | | |
| | 39 | 0.58 | 0 | 0.00E+00 | | | 1.51 | 2970 | 5900.66 | | |
| | 40 | 0.44 | 0 | 0.00E+00 | | | 1.48 | 2300 | 4662.16 | | |
| | 41 | 0.43 | 0 | 0.00E+00 | | | 1.72 | 1100 | 1918.60 | | |
| | 42 | 0.42 | 3.3 | 2.36E+01 | | | 1.46 | 1900 | 3904.11 | | |
| | 43 | 0.43 | 0 | 0.00E+00 | 0.47 | 0.036 | 1.31 | 1500 | 3435.11 | 6318.36 | 0.003 |

As can be seen from the Table for lung tissues, rats receiving DS) 7 showed reduced viral titers compared to B12 at all concentrations tested, and titer was generally lower in nasal tissues, especially at the highest concentration of DSλ7. These data correlate well with the protective immunity and antibody response data above (i.e., FIG. 8).

These data show that in animals that are permissive for HMPV replication in the nose and lungs can be used to model neutralization in vivo, and indicate that Fab clones (e.g., DSλ7) obtained by the methods as disclosed can reduce viral load in the lungs of these animals.

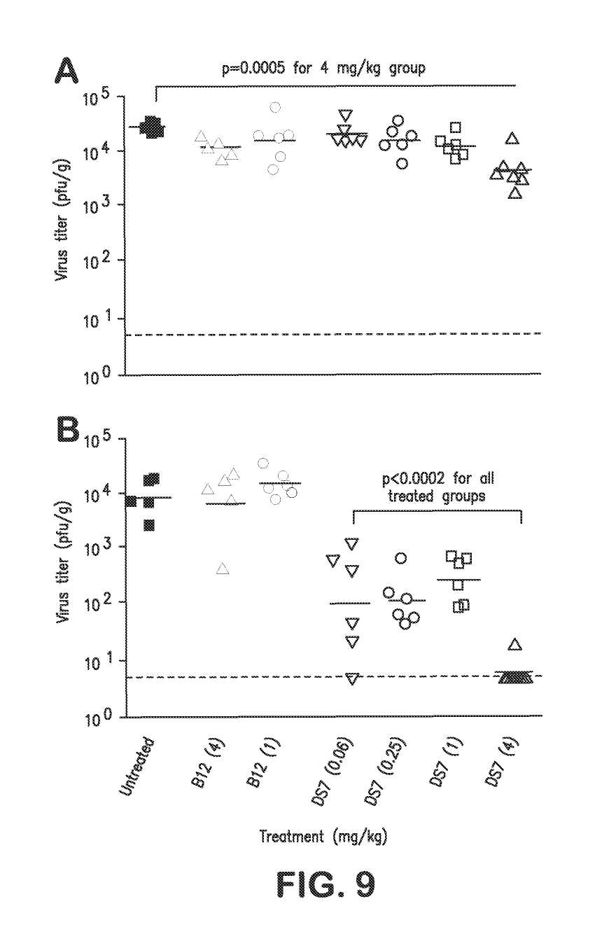

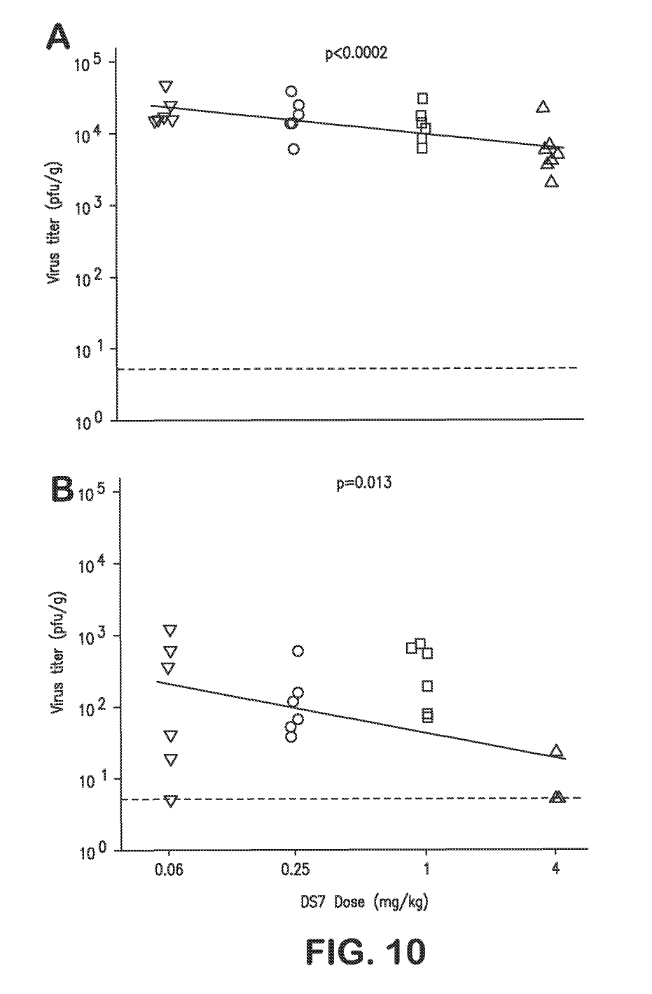

In another set of experiments, cotton rats were infected intranasally with HMPV and administered Fab intranasally on day 4, on day prior to the peak of HMPV replication (Williamson et al., J Virol (2005) 79:10944-10951). Animals were sacrificed and tissues harvested on day 4 and nasal and lung virus titers were determined. The virus titer in nasal turbinates was reduced only modestly by Fab DSλ7 treatment (FIG. 9A). There was a significant difference in virus titers between the control group (p=0.025), with those in the untreated control having a slightly higher geometric mean nasal titer than the two Fab B12-treated control groups (1.9×10⁴ PFU/g, HMPV; 1.2×10⁴ PFU/g, B12 4 mg/kg; 1.7×10⁴ PFU/g, B12 1 mg/kg). The DSλ7-treated groups to the combined control groups, and only the 4 mg/kg DSλ7 dose was associated with a significant reduction in nasal virus titer (4.9×10³ PFU/g vs. 1.8 10⁴ PFU/g, p=0.0005) (FIG. 9A). There was a statistically significant relationship between dose and response (p=0.0002): for every quadrupling of the dose, the expected viral load decreased approximately −0.20 log₁₀-PFU/g (95% CI of −0.29, −0.11) (FIG. 10A).

DSλ7 was highly effective at reducing viral titers in the lungs (FIG. 9B). The control groups (either untreated, or treated with Fab B12) had a mean lung virus titer of 9.6×10³ PFU/g. The lung virus titers did not differ between the three control groups (p=0.38). Each of the DST-treated groups had a lower lung virus titer than the controls (p<0.0002 for each group compared to controls). The mean virus titer in the lungs of DSλ7-treated animals ranged from 1.1×10² (0.06 mg/kg dose) to 6.2×10⁰ PFU/g (4 mg/kg dose). Only one of the seven animals in the DSλ7 4 mg/kg group had detectable virus in the lungs. This represented a >1500-fold reduction in the 4 mg/kg treated cotton rats compared to controls. There was evidence that higher doses resulted in lower lung virus titers (p=0.013; slope −0.36 log₁₀-PFU/g per dose quadrupling [95% CI of −0.62, −0.10]) (FIG. 10B). However, this result was driven by the 4 mg/kg dose. The was no evidence to suggest that the lung virus titer differed between 0.06, 0.25, and 1.0 mg/kg doses (p=0.37). Linear regression analysis was also performed without log₂-transforming the doses and the reduction in virus titer for both nasal and lung tissues was still significant (p<0.0001 for both; −0.62 Log₁₀-PFU/g [95% CI of −0.82, −0.43] for lung and −0.14 log₁₀-PFU/g [95% CI of −0.20, −0.09] for nasal turbinates).

Although the invention has been described with reference to the above examples, it will be understood that modifications and variations are encompassed within the spirit and scope of the invention. Accordingly, the invention is limited only by the following claims.

We claim:

1. An isolated human antibody that specifically binds to a human metapneumovirus (HMPV) fusion glycoprotein (F-protein).

2. The isolated antibody of claim 1, wherein the F-protein is selected from the group consisting of an amino acid sequence as set forth in SEQ ID NO:30, SEQ ID NO:32, SEQ ID NO:34, and SEQ ID NO:36.

3. The isolated antibody of claim 1, wherein the antibody comprises an HCDR3 amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

4. The isolated antibody of claim 1, wherein the antibody neutralizes HMPV genogroups A1, A2, B1, and B2.

5. The isolated antibody of claim 1, wherein antibody neutralizes HMPV genogroup A2.

6. The isolated antibody of claim 3, wherein the antibody comprises an HCRD3 amino acid sequence as set forth in SEQ ID NO:12.

7. The isolated antibody of claim 1, further comprising a detectable label.

8. The isolated antibody of claim 1, wherein the antibody is a humanized antibody.

9. The isolated antibody of claim 1, wherein the antibody is a CDR-grafted antibody.

10. The isolated antibody of claim 1, wherein the antibody is an antibody fragment.

11. The isolated antibody of claim 10, wherein the antibody fragment is an Fab, Fab′, F(ab′)₂, or Fc fragment.

12. The isolated antibody of claim 1, wherein the antibody is a monoclonal antibody.

13. A method for identifying a neutralizing antibody comprising:
i) generating a panel of antibodies against recombinant, immature, and mature forms of a fusion protein (F-protein);
ii) comparing the binding of the antibodies to each form of F-protein by competition analysis;
iii) determining the K_{d} for each antibody in the panel against each form of the F-protein;
iv) identifying one or more antibodies in the panel whose K_{d} is one or more orders of magnitude higher for the recombinant or immature form of the F-protein than the mature form of the F-protein; and
v) determining the neutralizing efficiency of the one or more antibodies identified in step (iv),
wherein a neutralizing antibody has lower binding constant for mature forms of the F-protein than a non-neutralizing antibody.

14. The method of claim 13, wherein the F-protein is selected from the group consisting of an amino acid sequence as set forth in SEQ ID NO:30, SEQ ID NO:32, SEQ ID NO:34, and SEQ ID NO:36.

15. The method of claim 13, wherein the antibody neutralizes HMPV genogroup A2.

16. The method of claim 15, wherein the antibody neutralizes HMPV genogroup A1 or B1.

17. A method of treating a respiratory condition in a subject, wherein the condition is caused by a human metapneumovirus (HMPV) infection, comprising administering to the subject an antibody which neutralizes HMPV.

18. The method of claim 17, wherein the antibody neutralizes HMPV genogroups A1, A2, B1, and B2.

19. The method of claim 18, wherein the antibody neutralizes HMPV genogroup A2.

20. The method of claim 19, wherein the antibody comprises an HCRD3 amino acid sequence as set forth in SEQ ID NO:12.

21. The method of claim 17, wherein the subject is a mammal.

22. The method of claim 21, wherein the mammal is a human.

23. A vaccine comprising one or more human antibodies that specifically binds to a human metapneumovirus (HMPV) fusion glycoprotein (F-protein).

24. The vaccine of claim 23, wherein the one or more antibodies comprise an HCDR3 amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, or SEQ ID NO:28.

25. A diagnostic kit for determining the presence of human metapneumovirus (HMPV) in a sample comprising:
a) a device for contacting a biological sample with one or more human antibodies that specifically bind to one or more HMPV fusion glycoproteins (F-proteins) under conditions that allow for the formation of a complex between the one or more antibodies and one or more HMPV F-proteins;
b) one or more reagents which remove non-complexed antibody;
c) one or more reagents that recognize the antibody;
d) instructions which provide procedures on the use of the antibody and reagents; and
e) a container which houses the one or more antibodies, reagents, and instructions.
